(19) United States
(12) Patent Application Publication
(10) Pub. No.: US 2009/0048179 A1
(43) Pub. Date: Feb. 19, 2009

(54) USE OF 5-PHOSPHODIESTERASE INHIBITORS TO ENHANCE THE PERMEABILITY OF THE BLOOD-BRAIN BARRIER OF ABNORMAL BRAIN TISSUE AND THE BLOOD-TUMOR BARRIER

(75) Inventor: Keith L. Black, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/238,872

(22) Filed: Sep. 26, 2008

Related U.S. Application Data
(63) Continuation-in-part of application No. 11/813,494, filed on Jul. 6, 2007.
(60) Provisional application No. 60/976,161, filed on Sep. 28, 2007.
(60) Provisional application No. 60/655,075, filed on Feb. 22, 2005.

(51) Int. Cl. A61K 38/08; A61K 31/519; A61K 31/4985; A61P 35/00; A61K 31/282

(52) U.S. Cl. 514/15; 514/252.16; 514/250; 514/34; 514/492

(57) ABSTRACT

This invention relates to methods and kits for enhancing the permeability of the blood-brain barrier of abnormal brain tissue or the blood-tumor barrier. Particularly, methods comprising the administration of 5-phosphodiesterase inhibitors, such as sildenafil and vardenafil, to selectively enhance the permeability of the blood-brain barrier of abnormal brain tissue or the blood-tumor barrier are described. This selective enhancement allows for selective delivery of therapeutic agents or imaging to treat the abnormal brain tissue or a tumor, including brain tumors and non-central nervous system tumors.

### CROSS-REFERENCE TO RELATED APPLICATIONS

The present application claims priority to U.S. Provisional Application Ser. No. 60/976,161, filed Sep. 28, 2007, and is also a continuation-in-part of U.S. application Ser. No. 11/813,494, filed Jul. 6, 2007, which is a national stage of International Application No. PCT/US06/05980, filed Feb. 22, 2006, which in turn claims priority to U.S. Provisional Application Ser. No. 60/655,075, filed Feb. 22, 2005, each of which are incorporated by reference herein in their entirety.

### FIELD OF INVENTION

The invention relates to methods for selectively enhancing the permeability of the blood-brain barrier of abnormal brain tissue and the blood-tumor barrier in a mammal.

### BACKGROUND OF THE INVENTION

It is known that a “blood-brain barrier” (“BBB”) prevents many substances from crossing capillaries in the brain and entering brain tissue. The walls of the blood vessels that carry blood into the brain form this barrier. More permeable blood vessels in other regions of the body allow numerous molecules to cross through to tissue, but the tight construction of the vessels in the head guards against brain entry. Blood gases, such as oxygen, and small nutritional molecules are among the few items that can readily cross the BBB. In fact, the BBB prevents most molecules that are not transported by specific transport systems in brain capillaries, or that are larger than about 200 Da and non-lipid soluble from crossing capillaries in the brain and entering brain tissue.

The delivery of drugs and other therapeutic agents to areas of disease within the brain is significantly impeded by the BBB. Indeed, it is believed that many of the therapeutic drugs that could be of benefit in neurological diseases are prevented from entering the brain in adequate concentrations because of the BBB. The efficacy of these drugs is thus significantly impeded or eliminated altogether. A method for selectively increasing delivery of therapeutic drugs and other agents to the diseased brain, including brain tumors, would be of great importance.

Accumulating research on animals and humans is helping researchers to devise methodologies by which to transport important therapeutic agents across the BBB. In accordance with one technique, therapeutic agents are latched onto molecules that are naturally able to cross the BBB; for example, docosahexaenoic acid (“DHA”). There is some evidence to suggest that, by doing so, a variety of molecules can be carried across the BBB. Another method involves “opening” the BBB; for instance, mannitol may be used to cause cells that line the vessel walls to shrink temporarily and allow a therapeutic agent to cross the BBB. This methodology, however, involves the entire central nervous system and is thus not confined to specific areas.

The prognosis for patients with malignant glioma remains poor (Davis et al., Survival rates in patients with primary malignant brain tumors stratified by patient age and tumor histological type: an analysis based on Surveillance, Epidemiology, and End Results (SEER) data, 1973-1991. J NEUROSURG. 1998; 88:1-10; Brandes et al., The role of chemotherapy in recurrent malignant glioma: an overview. CANCER INVEST. 1996; 14: 551-559; Hau et al., Pegylated liposomal doxorubicin-efficacy in patients with recurrent high-grade glioma. CANCER. 2004; 100: 1199-1207.) One of the barriers to effective treatment of malignant brain tumors is limited transport of antitumor therapeutics across brain tumor capillaries (Hau et al., Pegylated liposomal doxorubicin-efficacy in patients with recurrent high-grade glioma. CANCER. 2004; 100: 1199-1207; Balmaceda C., Advances in brain tumor chemosensitivity. CURR OPIN ONCOL. 1998; 10: 194-200.) Brain capillary endothelium and its contiguous cells, pericytes and astrocytes, are the structural and functional components of the BBB. The blood-tumor barrier (BTB), which includes the microvessels supplying brain tumors, retains many characteristics of the normal BBB that significantly impedes adequate delivery of chemotherapeutics into brain tumors (Black K L., The blood-brain barrier. In: J. R. Youmans (eds.), NEUROLOGICAL SURGERY. pp. Philadelphia: W.B. Saunders Co. 1996; 482-490; Groothuis, D R., The blood-brain and blood-tumor barriers: a review of strategies for increasing drug delivery. NEURO ONCOL. 2000; 2: 45-59; Neuwell et al., Effect of adrenal cortical steroids and osmotic blood-brain barrier opening on methotrexate delivery to glioma in the rodent: the factor of the blood brain barrier. PROC NATL ACAD SCI USA. 1982; 79: 4420-4423.) The BTB, however, has structural and functional characteristics that are different from those of the normal BBB. In particular, there are uniquely overexpressed receptors, ion channels and enzymes that regulate vascular permeability (Matsukado et al., Enhanced tumor uptake of carboplatin and survival in glioma-bearing rats by intracarotid infusion of bradykin analog, RMP-7. NEUROSURGERY. 1996; 39: 125-134; Nomura et al., Intracarotid infusion of bradykinin selectively increases blood-tumor permeability in 9L and C6 brain tumors. BRAIN RES. 1994; 659: 62-66; Inamura et al., Bradykinin selectively opens blood-tumor barrier in experimental brain tumors. J CEREB BLOOD FLOW METAB. 1994; 14: 862-870; Black, K L., Biochemical opening of the blood-brain barrier. ADV DRUG DELIVERY REV. 1995; 15: 37-52; Black et al., Intracarortid infusion of RMP-7, a bradylinin analog, increases transport of gallium-68 EDTA into human glioma. J NEUROSURG. 1997; 86: 603-609; Ningaraj et al., Regulation of blood-brain tumor barrier permeability by calcium-activated potassium channels. J PHARMACOLOGY AND EXPERIMENTAL THERAPEUTICS. 2002; 301: 838-851.) Pharmacologically modulating these unique components of the BTB can selectively increase transport of anti-tumor therapeutics across tumor capillaries and into brain tumors. For example, it was previously shown that bradykinin (BK) and its analog, RMP-7, an agonist to the bradykinin-2 receptor (B2R), selectively increase drug transport across the BTB in rat brain tumor models and humans (Matsukado et al., Enhanced tumor uptake of Carboplatin and survival in glioma-bearing rats by intracarotid infusion of bradykin analog, RMP-7. NEUROSURGERY. 1996; 39: 125-134; Nomura et al., Intracarotid infusion of bradykinin selectively increases blood-tumor permeability in 9L and C6 brain tumors. BRAIN RES. 1994; 659: 62-66; Inamura et al., Bradykinin selectively opens blood-tumor barrier in experimental brain tumors. J CEREB BLOOD FLOW METAB. 1994; 14: 862-870; Black, K L., Biochemical opening of the blood-brain barrier. ADV DRUG DELIVERY REV. 1995; 15: 37-52). However, increased transport across the BTB mediated by RMP-7 is transient and dependent on B2R expression in individual brain tumors (Nomura et al., Intracarotid infusion of bradykinin selectively increases blood-tumor permeability in 9L and C6 brain tumors. BRAIN RES. 1994; 659: 62-66; Inamura et al., Bradykinin selectively opens blood-tumor barrier in experimental brain tumors. J CEREB BLOOD FLOW METAB. 1994; 14: 862-870; Tsuboi et al., Elevated bradykinin and decreased carboxypeptidase R as a cause of hypotension during tryptophan column immunoabsorption therapy. THER APHER. 1998; 2: 297-299). High enough doses of RMP-7 were not achieved in phase II and III clinical trials to improve the efficacy of chemotherapy, due to the dose limiting side effect of hypotension (Liu et al., Correlation between bradykinin induced blood tumor barrier permeability and B2 receptor expression in experimental brain tumors. NEUROLOGICAL RES. 2001; 23: 379-387). Additionally, prolonged infusion of BK leads to B2R internalization and subsequent loss of the sensitivity of BTB transport in response to BK (Pizard et al., Bradykinin-induced internalization of the human B2 receptor requires phosphorylation of three serine and two threonine residues at its carboxyl tail. J BIOL CHEM. 1999; 274: 12738-12747). Thus, there is a need for the identification of additional molecular targets that could be utilized and compounds that can modulate the molecular targets to consistently and selectively modulate transport of therapeutics across the BTB.

### SUMMARY OF THE INVENTION

The present invention provides for methods to selectively enhance the permeability of the blood-brain barrier of abnormal brain tissue (“abnormal BBB”) and/or the blood-tumor barrier (“BTB”) in a mammal in need thereof, comprising: providing a 5-phosphodiesterase (“PDE5”) inhibitor; and administering the PDE5 inhibitor in an amount sufficient to selectively enhance the permeability of the abnormal BBB or the BTB. In a further embodiment, the method may comprise administering an additional agent that enhances the permeability of the abnormal BBB or the BTB, which may a bradykinin, a nitrogen oxide donor drug, a potassium channel agonist, analogs thereof, or salts thereof.

In one embodiment, the abnormal brain tissue is a brain tumor and the abnormal BBB and/or the BTB permeability is enhanced in the brain tumor. In another embodiment, the BTB permeability is enhanced in a non-central nervous system (“CNS”) tumor; for example, breast cancer or lung cancer. In various embodiments, the PDE5 inhibitor may be sildenafil, vardenafil, salts thereof, or analogs thereof. In various embodiments, the permeability of the abnormal BBB and/or the BTB is enhanced for at least 30 minutes, or for about 30 to about 360 minutes. In one embodiment, the PDE5 inhibitor is orally administered. Particularly useful is to administer the PDE5 inhibitor prior to administering a therapeutic agent or an imaging agent; for example, about 30 to about 180 minutes prior to administering the therapeutic agent or the imaging agent, or about 30 to about 45 minutes prior to administering the therapeutic agent or the imaging agent. In one embodiment, the therapeutic agent may be an anti-cancer drug; for example, doxorubicin or carboplatin.

The present invention also provides for methods for treating abnormal brain tissue and/or a tumor in a mammal in need thereof, comprising: providing a 5-phosphodiesterase (“PDE5”) inhibitor; administering the PDE5 inhibitor in an amount sufficient to selectively enhance the permeability of the blood-brain barrier of abnormal brain tissue (“abnormal BBB”) and/or the blood-tumor barrier (“BTB”) in the mammal; and administering a therapeutic agent to the mammal. In a further embodiment, the method may also comprise administering an additional agent that enhances the permeability of the abnormal BBB and/or the BTB, which may be a bradykinin, a nitrogen oxide donor drug, a potassium channel agonist, salts thereof, or analogs thereof.

In one embodiment, the abnormal brain tissue may be a brain tumor, a tissue affected by a disease, or a tissue affected by a physical injury. In various embodiments, the brain tumor may be a glioma, glioblastoma, glioblastoma multiforme, gliosarcoma, oligodendroglioma, primitive neuroectodermal tumor, astrocytoma, ependymoma, oligodendroglioma, medulloblastoma, meningioma, pituitary adenomas, neuroblastoma, or craniopharyngioma. In another embodiment, the tumor may be a non-central nervous system (“CNS”) tumor; for example, breast cancer or lung cancer.

The PDE5 inhibitor used may be sildenafil, vardenafil, salts thereof, or analogs thereof. In one embodiment, the PDE5 inhibitor is administered prior to administering the therapeutic agent; for example, about 30 to about 180 minutes prior to administering the therapeutic agent, or about 30 to about 45 minutes prior to administering the therapeutic agent. In a particular embodiment, the PDE5 inhibitor may be orally administered. In various embodiments, the therapeutic agent may be doxorubicin or carboplatin.

The present invention also provides for a kit for the treatment of a tumor in a mammal in need thereof, comprising: a quantity of a PDE5 inhibitor to selectively enhance the permeability of the blood-brain barrier of abnormal brain tissue (“abnormal BBB”) and/or the blood-tumor barrier (“BTB”); a quantity of an anti-cancer drug; and instructions for using the PDE5 inhibitor to selectively enhance the permeability of the abnormal BBB and/or the BTB to the anti-cancer drug to treat the tumor.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are considered illustrative rather than restrictive.

FIG. 1 depicts the dose response effect of PDE5 inhibitors on tumor permeability in accordance with various embodiments of the present invention. PDE5 inhibitors were administered orally at various doses followed by permeability determination at the 50′-60′ time interval. Bradykinin was infused for 15′ with permeability determined at the 5′-15′ time interval. (A) Viagra® (sildenafil). (B) Levitra® (vardenafil). (C) Cialis® (tadalafil). Error bars=SEM.

FIG. 2 depicts the time course of oral PDE % inhibitors on tumor permeability in accordance with various embodiments of the present invention. PDE5 inhibitors were administered orally followed by permeability determination at varying time points. (A) Viagra® (sildenafil). (B) Levitra® (vardenafil). C, Clalis® (tadalafil). Error bars=SEM.

FIG. 3 depicts the time course of oral PDE5 inhibitor and bradykinin combinations on tumor permeability in accordance with various embodiments of the present invention. PDE5 inhibitors ((A) Viagra® (sildenafil) 50 mg/kg; (B) Levitra® (vardenafil) 10 mg/kg) were administered orally followed bradykinin infusion for the final 15 minutes followed by permeability determination at varying time points. Error bars=SEM.

FIG. 4 depicts the effect of Levitra® (vardenafil) and Adriamycin® (doxorubicin) on the survival in 9L/Fischer Rats in accordance with various embodiments of the present invention. The rats were given treatment on days 4, 5 and 6 post tumor implantation. The study was in progress and the data depicted represents the study as of Feb. 15, 2006. (A) Survival Rate; (B) Survival Days.

FIGS. 5A and 5B depict the effect of Levitra® (vardenafil) and Adriamycin® (doxorubicin) on the survival in 9L/Fischer Rats in accordance with various embodiments of the present invention. The rats were given treatment on days 7, 8 and 9 post tumor implantation.

FIG. 6 depicts the effect of Levitra® (Vardenafil) on cGMP levels in plasma from 9L glioma Fischer rats in accordance with an embodiment of the present invention.

FIG. 7 depicts cyclic GMP expression in brains of 9L glioma bearing rats as detected by immunocytochemistry in accordance with an embodiment of the present invention.

FIG. 8 depicts expression of PDE1, PDE5 and PDE10 in 9L gliosarcoma cells in accordance with various embodiments of the present invention. (A) Amplication of the three PDEs by real-time PCR; (B) The mRNA levels of the three PDEs normalized to GAPDH. Higher levels of PDE5 mRNA were detected compared to those of PDE1 and PDE10.

FIG. 9 depicts pharmacological modulation of tumor permeability by PDE5 inhibitors in accordance with various embodiments of the present invention. (A) Effects of oral PDE5 inhibitors on the rate of radioactive sucrose transport, Ki, into tumors. The PDE5 inhibitors were administered orally followed by permeability determination at the 60-minute time point. BK (120 μg/kg/min) was infused for 15 minutes with permeability determined at the 15-minute time point. Intravenous infusion of BK served as a positive control. (B) Effect of a selective K_{Ca} channel antagonist, iberiotoxin (IBTX), on transport induced by vardenafil. IBTX (0.26 μg/kg/min) was infused for 15 minutes with permeability determined during the 5 to 15-minute infusion interval. The data are presented as mean±SEM. SIL, sildenafil (50 mg/kg); VAR, vardenafil (10 mg/kg). * p<0.001, significantly different from the saline-treated group.

FIG. 10 depicts time course of effects of oral PDE5 inhibitors on tracer transport into tumors in accordance with various embodiments of the present invention. (A) sildenafil treatment (50 mg/kg); (B) vardenafil treatment (10 mg/kg). The PDE5 inhibitors were administered orally followed by transport determination at various time points. The data are presented as mean±SEM. SIL, sildenafil; VAR, vardenafil. * p<0.05, ** p<0.01, and *** p<0.001, significantly different from the saline-treated group.

FIG. 11 depicts the effect of the combination treatment with oral PDE5 inhibitors and intravenous BK infusion on transport into tumors in accordance with various embodiments of the present invention. (A) sildenafil with or without BK; (B) the permeability at different brain areas by the combination treatment. The PDE5 inhibitor sildenafil (50 mg/kg) were administered by gavage at different time points with or without BK infusion (120 μg/kg/min for 15 minutes). BK, bradykinin; SIL, sildenafil; VAR, vardenafil; Cortex-lps, ipsilateral cortex, Cortex-Contra, contralateral cortex. The data are presented as mean±SEM. *** p<0.001, significantly different from saline control group. TTTp <0.001, significantly different from BK-treated group. φφ p<0.01, significantly different from sildenafil-treated group.

FIG. 12 depicts the effect of vardenafil on the cyclic GMP (cGMP) levels in the plasma and tissues in accordance with various embodiments of the present invention. (A) Effect of vardenafil on cGMP levels in the plasma of rats. Vardenafil (10 mg/kg) was administered orally and blood collected at 30, 60, 90 and 120 minutes later. cGMP levels were determined by ELISA-based analysis. (B) Effect of vardenafil on cGMP levels in the 9L tumor. cGMP was detected by immunocytochemistry. Scale bar, 240 μm. Brain tumors were removed 30, 60, 90 and 120 minutes after oral administration of vardenafil (10 mg/kg). The representative imagines for tumor and the normal tissue contralateral to the tumor (60 minutes after vardenafil) are also shown. Fluorescent microscopy was performed using anti-cGMP primary and FITC-conjugated secondary antibodies. (C) Semi-quantification of immunofluorescent intensity of cGMP staining in brain and tumor tissues. The data are presented as mean±SEM. VAR, vardenafil. * p<0.05, significantly different from the control group.

FIG. 13 depicts that vardenafil treatment increases vesicle formation in brain tumor capillary endothelium in accordance with various embodiments of the present invention. Representative electron microscopic photographs of endothelium from the treatments with PBS ((a) normal tissue; (b) tumor tissue) and vardenafil (tumor tissue; (c) 1 hour; (d) 2 hours; (e) 3 hours after treatment) are shown. Treatment with vardenafil significantly increase the number of vesicles in the endothelial cytoplasm (arrowheads) compared to PBS treatment. Original magnification, 60,000×.

FIG. 14 also depicts that vardenafil treatment increases vesicle formation in brain tumor capillary endothelium in accordance with various embodiments of the present invention. Vesicular density of the endothelial cytoplasm is shown (number of vesicles/μm²). Note that vesicular density is significantly increased after vardenafil treatment compared with control. *p<0.01, **p<0.001 compared with the PBS control of tumor samples.

FIG. 15 depicts morphometric evaluation of the degree of opening of the tight junctions in endothelial cells from different treatments in accordance with various embodiments of the present invention. Representative electron microscopic photographs of endothelium treated with PBS ((a) normal; (b) tumor) and vardenafil (tumor tissue; (c) 1 hour; (d) 2 hours; (e) 3 hours after treatment) are shown. The dark gray arrows indicate tight junction and white arrows indicate a cleft of tight junction.

FIGS. 16A and 16B also depict morphometric evaluation of the degree of opening of the tight junctions in endothelial cells from different treatments in accordance with various embodiments of the present invention. (A) Cleft index (% of increase) measurement of tight junction in endothelial cells from different treatments. (B) Cleft area index (%) measurement of tight junction in endothelial cells from different treatments. Cleft morphology (cleft index and cleft area) was altered in BTB capillaries compared with (normal) BBB capillaries, but was not changed as a result of vardenafil treatment.

FIG. 17 depicts the effect of vardenafil on Adriamycin® (doxorubicin) chemotherapy in the 9L gliosarcoma-bearing rats in accordance with various embodiments of the present invention. (A) Effect of vardenafil on the survival rates of the 9L gliosarcoma-bearing rats. The rats were treated with saline, vardenafil (10 mg/kg, oral), Adriamycin® (doxorubicin) (2 mg/kg, iv), or vardenafil (10 mg/kg, oral) plus Adriamycin® (doxorubicin) (2 mg/kg, iv). All rats were treated for three consecutive days beginning on the fourth day after tumor implantation (1×10⁵ 9L cells/rat). The Kaplan-Meier survival curves showed that the rats treated with vardenafil plus Adriamycin® (doxorubicin) survived significantly (*p<0.05) longer than other three groups of rats. The rats treated with Adriamycin® (doxorubicin) alone also survived significantly (*p<0.05) longer than the untreated and the vardenafil alone-treated rats. VAR, vardenafil. The data are presented as mean±SEM. (B) Effect of vardenafil on tumor size in the 9L gliosarcoma-bearing rats. Rats were implanted with 9L tumor cells containing the luciferase gene and treated with Adriamycin® (doxorubicin) (2 mg/kg, iv) with and without vardenafil treatment (10 mg/kg, oral) on days 4, 5 and 6 following implantation. Bioluminescence imaging was performed 9 days after the last treatment using the Xenogen IVIS 200 imaging system. Representative animals from the four groups of rats are shown.

FIG. 18 depicts in vitro cellular transport studies in accordance with various embodiments of the present invention. (A) Sildenafil significantly increased ¹⁴C-sucrose transport in mouse brain endothelial cells. *P<0.05 for the difference between two treatments. (B) In vitro studies in the vessel endothelial cells derived from lung tissues (PVEC). Sildenafil also significantly increased ¹⁴C-sucrose transport in PVECs.

FIG. 19 depicts inhibition of PDE5 enhances tracer transport in different tumor cell lines in accordance with various embodiments of the present invention. Two tumor cell lines (human brain metastatic lung cancer cell line CRL-5904 and human breast cancer cell line MDA-MB-231) were tested. Sildenafil increased ¹⁴C-sucrose transport across both cell lines seeded in the transwell (A & B).

FIG. 20 depicts inhibition of PDE5 enhances chemotherapeutics transport in endothelial and/or tumor cells in accordance with various embodiments of the present invention. ¹⁴C-sucrose was used to examine the general permeability. In vitro cellular transport studies to determine whether the inhibition of PDE5 enhance the transport of chemotherapeutics were also performed (*P<0.05 compared with doxorubicin alone). Two hydrophilic chemotherapeutics, doxorubicin and carboplatin, were used. Vardenalfil significantly enhanced Adriamycin® (doxorubicin) transport in human brain endothelial cells (A) and CRL-5904 tumor cells (B).

FIG. 21 depicts filipin, an inhibitor of caveolae endocytosis, abolishing the increased transport of ¹⁴C-sucrose by sildenafil in the in vitro cellular transport study (P <0.05 vs. other two treatments) in accordance with various embodiments of the present invention.

FIG. 22 depicts Levitra® (vardenafil) treatment increasing the fluorescence intensity in both brain and flank tumors (from CRL-5904 cells) in accordance with various embodiments of the present invention. Further, a more dramatic increase was observed for (A) mbrain tumors (from CRL-5904 cells) as compared with (B) flank tumors.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Abnormal brain tissue” as used herein refers to brain tissue characterized by abnormal cell proliferation; for example, gliomas, glioblastomas, glioblastoma multiforme (GBM), gliosarcoma, oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, and craniopharyngiomas. Abnormal brain tissue also refers to brain tissue physiologically affected by physical injury (e.g., trauma) or biochemical injury. Examples of diseases that may assert biochemical injury include: degenerative brain disease, cerebrovascular disease (e.g., stroke, embolic stroke), cerebral ischemia, infection, migraine, convulsion, bacterial infection, viral infection (e.g., HIV infection), schizophrenia, Parkinson's, Alzheimer's, hypoxia, cerebral palsy, dyspnea, encephalopathy, meningitis, cerebral abscess, multiple sclerosis, and subarachnoid hemorrhage.

“Cancer” and “cancerous” refer to or describe the physiological condition in mammals that is typically characterized by unregulated cell growth. Examples of cancer include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer; including, but not limited to, gliomas, glioblastomas, glioblastoma multiforme (GBM), gliosarcomas, oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, malignant meningiomas, pituitary carcinomas, neuroblastomas, and craniopharyngiomas.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

“Therapeutically effective amount” as used herein refers to that amount which is capable of measurably enhancing permeability of a reference molecule across the abnormal BBB and/or BTB. A therapeutically effective amount can be determined on an individual basis and will be based, at least in part, on consideration of the physiological characteristics of the mammal, the degree of abnormality of BBB permeability in that mammal, and the properties of the reference molecule that is targeted for enhanced transport across the abnormal BBB and/or BTB.

“Treatment” and “treating,” as used herein refer to both therapeutic treatment and prophylactic or preventative measures, wherein the object is to prevent or slow down (lessen) the targeted pathologic condition or disorder even if the treatment is ultimately unsuccessful. Those in need of treatment include those already with the disorder as well as those prone to have the disorder or those in whom the disorder is to be prevented. For example, in tumor (e.g., cancer) treatment, a therapeutic agent may directly decrease the pathology of tumor cells, or render the tumor cells more susceptible to treatment by other therapeutic agents.

“Therapeutic agent” as used herein refers to agents capable of treating abnormal brain tissue; for example, chemotherapeutic drugs for treatment of brain tumors. Additional examples of therapeutic agents include: anti-cancer drugs, therapeutic viral particles, antiproliferative agents, antimicrobials (e.g., antibiotics, antifungals, antivirals), mood-stabilizing agents, anticonvulsants, anti-neurodegenerative agents, anti-stroke agents, cytokines, therapeutic proteins, immunotoxins, immunosuppressants, and gene therapeutics (e.g., adenoviral vectors, adeno-associated viral vectors, retroviral vectors, herpes simplex viral vectors, pox virus vectors). Other suitable therapeutic agents will be readily recognized by those of skill in the art.

“Tumor,” as used herein refers to all neoplastic cell growth and proliferation, whether malignant or benign, and all pre-cancerous and cancerous cells and tissues.

Described herein, the inventor sought to determine whether PDE5 inhibitors could increase the permeability of the blood-tumor barrier (“BTB”) and the blood-brain barrier of abnormal brain tissue (also referred to as “abnormal BBB”) and thereby improve the efficacy of chemotherapeutic treatment of brain tumors, non-central nervous system (CNS) tumors, and abnormal brain tissue (e.g., tissue physiologically affected by physical injury or biochemical injury). Cyclic guanosine monophosphate (cGMP) is an important intracellular second messenger that has been implicated in the regulation of vascular tone and permeability (Murad, F., Regulation of cytosolic guanylyl cyclase by nitric oxide: the NO-cGMP signal transduction system. AVD PHARMACOL. 1994; 26: 19-33; 18; Sharma et al., Vasoactive substances induce cytoskeletal changes in cultured rat glomerular epithelial cells. J AM SOC NEPHROL. 1992; 3: 1131-1138; Ignarro et al., Endothelium-dependent modulation of cGMP levels and intrinsic smooth muscle tone in isolated bovine intrapulmonary artery and vein. CIRC RES. 1987; 60: 82-92). cGMP is made from GTP in a reaction catalyzed by guanylyl cyclases, and is degraded to 5′-GMP by phosphodiesterases (PDE) (Beavo, J A., Cyclic neuleotide phosphodiesterases: functional implications of multiple isoforme. PHYSIOL REV. 1995; 75: 725-745; Bentley et al., Regulation and function of cyclic nucleotides. CURR OPIN CELL BIOL. 1992; 4: 233-240). Modulation of PDE activity, which can affect the levels of intracellular cGMP, may result in alteration of the permeability of capillaries including microvessels in brain tumors. (Sugita et al., Cyclic GMP-specific phosphodiesterase inhibition and intracarotid bradykinin infusion enhance permeability into brain tumors. CANCER RES. 1998; 58: 914-920.) Sildenafil (Viagra®), vardenafil (Levitra®) are selective inhibitors of type 5 PDE (PDE5) that increase intracellular cGMP levels (Corbin et al., Cyclic GMP phosphodiesterase-5: target of sildenafil. J BIOL CHEM. 1999; 274: 13729-13732; Corbin et al., Vardenafil: structural basis for higher potency over sildenafil in inhibiting cGMP-specific phosphodiesterase-5 (PDE5). NEUROCHEMISTRY INTERNATIONAL. 2004; 45: 859-863) and are FDA approved oral treatments for erectile dysfunction in men.

In this study, the inventor tested the whether cGMP signaling is involved in the transport of compounds across the BTB. In particular, the inventor investigated whether oral PDE5 inhibitors at doses well tolerated in humans could selectively increase BTB permeability in rat brain tumor models. The ultrastructure of brain tumor capillaries after PDE5 inhibitor treatment was also examined. Further, the inventor examined whether oral PDE5 inhibitors given in combination with a chemotherapeutic could improve the survival of the animals bearing a malignant glial tumor. The findings described herein support the use of PDE5 inhibitors as a novel therapy to selectively increase drug transport to tumors, including malignant brain tumors and non-CNS tumors, as well as abnormal brain tissue caused by diseases.

The present invention involves the use of 5-phosphodiesterase (“PDE5”) inhibitors to enhance the permeability of the BTB and the abnormal BBB and thereby enhance the transport of compounds (e.g., therapeutic agents, imaging agents) through the blood vessels (e.g., capillaries) present in tumors and abnormal brain tissue. Thus, the present invention describes compositions, methods and kits to enhance the permeability of the BTB and the permeability of the abnormal BBB to various compounds. One particular embodiment, the invention relates to the BTB of cancerous tumors. This invention is enabled by the inventor's work as described herein.

The inventor sought to understand whether cGMP signaling is involved in determining the rate of transport of compounds across the BTB and particularly whether the pathway could be modulated by inhibition of PDEs, which are key enzymes determining intracellular cGMP levels, to improve efficacy of chemotherapy for brain tumors. The inventor observed that PDE5 is highly expressed in 9L tumor cells, brain capillary endothelial cells and tumor cell lines. Oral administration of the PDE5 inhibitors vardenafil and sildenafil selectively increased cGMP levels and vesicular transport in tumors and increased the rate of transport of [¹⁴C] sucrose, a radioactive trace, from blood to brain tumor tissue. Importantly, vardenafil, when given in combination with Adriamycin® (doxorubicin), significantly improved the survival and reduced the tumor size in brain-tumor bearing rats. Collectively, the inventor demonstrated that oral administration of PDE5 inhibitors such as vardenafil and sildenafil increased the rate of transport of compounds across the BTB and improved the efficacy of Adriamycin® (doxorubicin) in treatment of brain tumors in a rat model. Previously, the inventor demonstrated that bradykinin (“BK”) increases transport across the BTB in rat brain tumor models via a mechanism involving cGMP (Sugita et al., Cyclic GMP-specific phosphodiesterase inhibition and intracarotid bradykinin infusion enhance permeability into brain tumors. CANCER RES. 1998; 58: 914-920) which is made from GTP in a reaction catalyzed by guanylyl cyclases and is degraded to 5′-GMP by PDEs (Beavo, J A., Cyclic neuleotide phosphodiesterases: functional implications of multiple isoforme. PHYSIOL REV. 1995; 75: 725-745; Bentley et al., Regulation and function of cyclic nucleotides. CURR OPIN CELL BIOL. 1992; 4: 233-240). Thus, the inventor believed that inhibition of PDEs may be utilized to increase BTB permeability. Currently, there are more than 11 PDE isoforms identified, most of which have been detected in the brain (Menniti et al., Phosphodiesterases in the CNS: targets for drug development. NAT. REV. DRUG. DISCOV. 2006; 5: 660-670). The inventor detected PDE5 mRNA in 9L tumor cells that were used to generate the brain tumor model in the experiments described herein, with less expression of PDE1 and PDE10. In addition, PDE5 expression was also detected in a human microvessel endothelial cell line, other brain tumor cell lines such as GL26, U87, and RG2, and human brain tumor samples available in the inventor's laboratory (data not shown). The inventor's findings suggest that PDE5 and other PDEs could serve as drug targets for selectively increasing transport of chemotherapeutic agents across the BTB.

Sildenafil and vardenafil are oral drugs that are currently used to treat erectile dysfunction in men. They selectively inhibit the activity of cGMP-specific PDE5 with different potencies (Corbin et al., Vardenafil: structural basis for higher protency over sildenafil in inhibiting cGMP-specific phosphodiesterase-5 (PDE5). NEUROCHEMISTRY INTERNATIONAL. 2004; 45: 859-863). Demonstrated herein, oral administration of vardenafil and sildenafil selectively increased transport of [¹⁴C] sucrose to brain tumors. Vardenafil appeared to have a greater effect than sildenafil. This may reflect a higher potency for vardenafil to inhibit PDE5 as described in a previous study (Corbin et al., Vardenafil: structural basis for higher protency over sildenafil in inhibiting cGMP-specific phosphodiesterase-5 (PDE5). NEUROCHEMISTRY INTERNATIONAL. 2004; 45: 859-863). It should be noted that the doses of sildenafil and vardenafil used in the animals were comparable to the dose range clinically approved for erectile dysfunction in men, and they did not result in any detectable side effects in the rats. The two drugs appeared to be at least if not more effective in increasing drug transport into tumors as compared to the infusion of high dose BK, which caused significant hypotension in rats (30% decrease in mean arterial blood pressure). In the experiments described herein, the inventor used 1000-times the BK dose used in previous human studies. The 1000-times dose cannot be used in human subjects because of hypotension. Since the use of the PDE5 inhibitors did not result in detectable side effects (e.g., hypotension), the use of the PDE5 inhibitors offers significant improvement over the prior art methods of enhancing the abnormal BBB and/or BTB.

Neither sildenafil nor vardenafil increased the transport of tracers into normal brain, thereby reducing potential toxicity of increased delivery of chemotherapeutic drugs to normal brain tissue. BK increases transport into tumor in rat brain tumor models (Matsukado et al., Enhanced tumor uptake of carboplatin and survival in glioma-bearing rats by intracarotid infusion of bradykin analog, RMP-7. NEUROSURGERY. 1996; 39: 125-134; Nomura et al., Intracarotid infusion of bradykinin selectively increases blood-tumor permeability in 9L and C6 brain tumors. BRAIN RES. 1994; 659: 62-66; Inamura et al., Bradykinin selectively opens blood-tumor barrier in experimental brain tumors. J CEREB BLOOD FLOW METAB. 1994; 14: 862-870). However, BK has a short biological half-life because of proteolytic inactivation (Nakano et al., Increased brain tumor microvessel permeability after intracarotid bradykinin infusion is mediated by nitric oxide and Ca²⁺-actuvated K⁺ channels. CIRCULATION. 1996; 99: 3132-3138; Anhoutte et al., Why are converting enzyme inhibitors vasodilators? BR J CLIN PHARMACOL. 1989; 28: 95S-104S) and its effect on transport into tumors is diminished within 15 minutes after intra-carotid infusion (Nomura et al., Intracarotid infusion of bradykinin selectively increases blood-tumor permeability in 9L and C6 brain tumors. BRAIN RES. 1994; 659: 62-66; Inamura et al., Bradykinin selectively opens blood-tumor barrier in experimental brain tumors. J CEREB BLOOD FLOW METAB. 1994; 14: 862-870), which makes the clinical use of BK or its analog RMP-7 difficult. The inventor found that transport of tracers into tumors remained elevated for a much longer period after oral administration of sildenafil or vardenafil in comparison to BK. The extended period of BTB opening by PDE5 inhibitors could facilitate a greater accumulation of anti-tumor therapeutic agents in malignant brain tumors when administration is optimized with the pharmacokinetics of the agents; a task well within the ordinary skill in the art and one that can be accomplished with merely routine experimentation. Thus, the use of PDE5 inhibitors can solve the problem of transient permeability discussed above.

In the absence of a PDE5 inhibitor, higher levels of cGMP were detected in brain tumors than in the normal brain, consistent with the higher baseline permeability in tumors. Oral administration of vardenafil further increased cGMP levels in tumor tissue. cGMP has been reported to be involved in determining brain tumor permeability (Sugita et al., Cyclic GMP-specific phosphodiesterase inhibition and intracarotid bradykinin infusion enhance permeability into brain tumors. CANCER RES. 1998; 58: 914-920). The inventor's data suggests that the increased rate of transport into tumor after treatment with PDE5 inhibitors is mediated via elevated cGMP levels. The observation that vardenafil elevated cGMP levels much more in the tumor tissue than in the normal brain is consistent with the selective effects of the drug on transfer rate (Ki) increase in tumors. In this study, the increased tumor permeability induced by PDE5 inhibitors could be abolished by a selective K_{Ca} channel antagonist, iberiotoxin. cGMP can activate cGMP-dependent protein kinase (PKG) which subsequently stimulates K_{Ca} channels (Erdos, E G., Some old and some new ideas on kinin metabolism. J CARDIOVASC PHARMACOL. 1990; 15: S20-S24). It has been reported in an animal brain tumor model that K_{Ca} channels are highly expressed in tumor microvessels compared to normal brain and their activation results in increased vesicular transport of drugs from blood to tumor tissue (Nakano et al., Increased brain tumor microvessel permeability after intracarotid bradykinin infusion is mediated by nitric oxide and Ca²⁺-actuvated K⁺ channels. CIRCULATION. 1996; 99: 3132-3138).

Three major cellular mechanisms have been suggested to account for increased BBB permeability: (1) increased vesicular transport, (2) increased opening of tight junctions of endothelial cells, and (3) increased transcellular penetration (Chan et al., Induction of brain edema following intracerebral injections of arachidonic acid. ANN NEURO. 1983; 13: 625-32; Stewart et al., Quantitation of blood-brain barrier ultrastructure. MICROSC RES TECH. 1994; 27: 516-27). The inventor investigated whether increased drug transport across the BTB by PDE5 inhibitors is associated with any change in vesicular transport and tight junction integrity. The data demonstrate that vardenafil treatment significantly increased vesicle formation without any effect on tight junction integrity in tumor capillary endothelium and without alteration of vesicular density in normal brain capillary endothelium. Further studies are required to determine the mechanism of transendothelial vesicular pathways mediated by vardenafil. For example, there are several transcytosis pathway in endothelium for substances from blood to brain such as receptor-mediated transport, carrier-mediated transport, and active efflux transport. Caveolae is most abundant in continuous capillary endothelia (Schnitzer et al., Albondin-mediated capillary permeability to albumin. Differential role of receptors in endothelial transcytosis and endocytosis of native and modified albumins. J BIOL CHEM. 1994; 269: 6072-82; Predescu et al., Functional and morphological studies of protein transcytosis in continuous endothelia. AM J PHYSIOL LUNG CELL MOL PHYSIOL. 2004; 287: L895-901; Sheikov et al., Brain arterioles show more active vesicular transport of blood-borne tracer molecules than capillaries and venules after focused ultrasound-evoked opening of the blood-brain barrier. ULTRASOUND MED BIOL. 2006; 32:1399-409) and could be a suitable means for the transfer of anti-cancer drugs or other therapeutics from blood to brain. (Frank et al., Caveolin, caveolae, and endothelial cell function. ARTERIOSCLER THROMB VASC BIOL. 2003; 23: 1161-8; Comford et al., Localization of brain endothelial luminal and abluminal transporters with immunogold electron microscopy. NEURORX. 2005; 2: 27-43). Filipin, caveolae-mediated vesicle inhibitor, can be used to determine whether vardenafil mediate caveolae dependent vesicular formation. Adriamycin® (doxorubicin) was the anti-tumor agent used in the experiments described herein. Although Adriamycin® (doxorubicin) is one of the most effective agents against brain tumor cell lines in vitro, (Wolff et al., Chemosensitivity of glioma cells in vitro: a meta analysis. J CANCER RES CLIN ONCOL. 1999; 125:481-486) it has little effect in vivo as the BTB limits its ability to cross the BTB in brain tumor-bearing rodent models (Hau et al., Pegylated liposomal doxorubicin-efficacy in patients with recurrent high-grade glioma. CANCER. 2004; 100:1199-1207; Steiniger et al., Chemotherapy of glioblastoma in rats using doxorubicin-loaded nanoparticles. INT J CANCER. 2004; 109: 759-767) in patients (Smith et al., Multilevel therapeutic targeting by topoisomerase inhibitors. BR J CANCER. 2004; 23: 547-550). However, a significant increase in survival rate was achieved with intratumoral injection of Adriamycin® (doxorubicin) in patients with malignant glioma (Voulgaris et al., Intratumoral doxorubicin in patients with malignant brain tumor. AM J CLIN ONCO. 2002; 25: 60-64; Walter et al., Intratumoral chemotherapy. NEUROSURGY. 1995; 37: 1128-1145). In the survival study, the treatment with a combination of oral vardenafil and Adriamycin® (doxorubicin) resulted in longer survival and smaller tumor size in the rats with a gliosarcoma. These results may be extended to other chemotherapeutics. It has been shown that sildenafil increases angiogenesis in the ischemic regions of the brain of rats (Zhang et al., Sildenafil (Viagra) induces neurogenesis and promotes functional recovery after stroke in rats. Stroke. 2002; 33: 2675-2680). The survival study showed that there is no difference in the survival days in control rats and rats treated with vardenafil, indicating vardenafil may not increase tumor infiltration. However, further studies need to be performed to find out whether PDE5 inhibitors could induce neovascularization in the brain tumor. [¹⁴C] sucrose, the tracer used for Ki measurement, is similar in molecular weight (MW, 342.3), water solubility and limited or lack of ability to cross the BTB as many of the chemotherapeutics currently used to treat human tumors. As such the ability of the BTB to become more permeable to the tracer, [¹⁴C] sucrose, indicates that the BTB will also become more permeable to many of the chemotherapeutics currently used to treat human tumors that are similar in molecular weight and water solubility. One of skill in the art can readily recognize and determine chemotherapeutics that are similar in molecular weight and water solubility without undue experimentation. The inventor has previously reported similar KI changes after biochemical modulation of the BTB for sucrose, carboplatin and methotrexate (Ningaraj et al., Regulation of blood-brain tumor barrier permeability by calcium-activated potassium channels. J Pharmacology and Experimental Therapeutics. 2002; 301: 838-851; Ningaraj et al., Adenosine 5′-triphosphate-sensitive potassium channel-mediated blood-brain tumor barrier permeability increase in a rat brain tumor model. CANCER RES. 2003; 63: 8899-88911; Inamura et al., Intracarotid histamine infusion increases blood tumor permeability in RG2 glioma. NEUROL RES. 1994; 16: 125-128). The water soluble Adriamycin® (doxorubicin) has a molecular weight (579.98) comparable to carboplatin (371.25) and methotrexate (454.44).

In conclusion, oral administration of PDE5 inhibitors selectively increases transport across brain tumor capillaries and enhances the efficacy of chemotherapy in a rat brain tumor model.

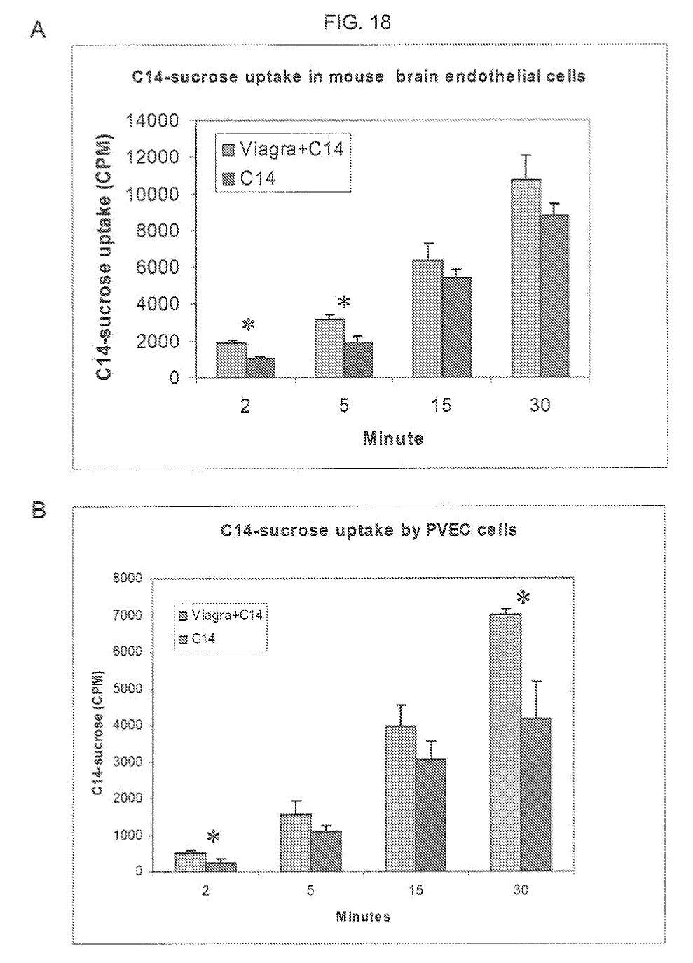

The inventor's studies also indicated that the inhibition of PDE5 also enhances tracer transport in vessel endothelial cell lines of different tissue origins. Accordingly, the administration of PDE5 inhibitors can also enhance the delivery of compounds to non central nervous system tumors. The inventor found that the PDE5 inhibitors vardenafil and sildenafil enhanced brain tumor permeability to the radiolabeled tracer ¹⁴C-sucrose in rats. To determine the molecular and cellular mechanisms of the enhanced permeability, in vitro cellular transport studies using brain vessel endothelial cell lines were performed (FIG. 18A; *P<0.05 for the difference between two treatments, same in the following figures). As shown, sildenafil significantly increased ¹⁴C-sucrose transport in mouse brain endothelial cells. The data is consistent with the increased brain tumor permeability by PDE5 inhibition in animals. Furthermore, to determine whether the enhancement of permeability by PDE5 inhibition has any general implication, similar in vitro studies were conducted in the vessel endothelial cells derived from lung tissues (PVEC) (FIG. 18B). Sildenafil also significantly increased ¹⁴C-sucrose transport in PVECs, indicating that PDE5 inhibition can increase the permeability of non-CNS tumors.

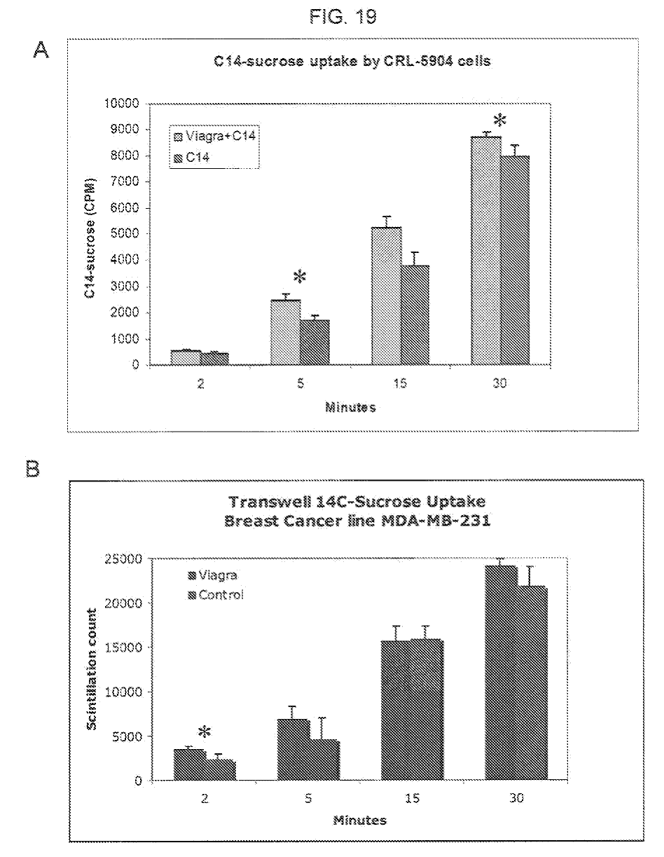

Studies also indicated that the inhibition of PDE5 enhances tracer transport in different tumor cell lines. In addition to endothelial cells, the inventor tested whether the inhibition of PDE5 has any effect on tracer transport in other tumor cells. In the studies, two tumor cell lines (human brain metastatic lung cancer cell line CRL-5904 and human breast cancer cell line MDA-MB-231) were tested. As shown, sildenafil increased ¹⁴C-sucrose transport across both cell lines seeded in the transwell (FIGS. 19A & 19B). The data is consistent with the ability of PDE5 inhibitors to increase not only brain tumor permeability but also those of peripheral tumors.

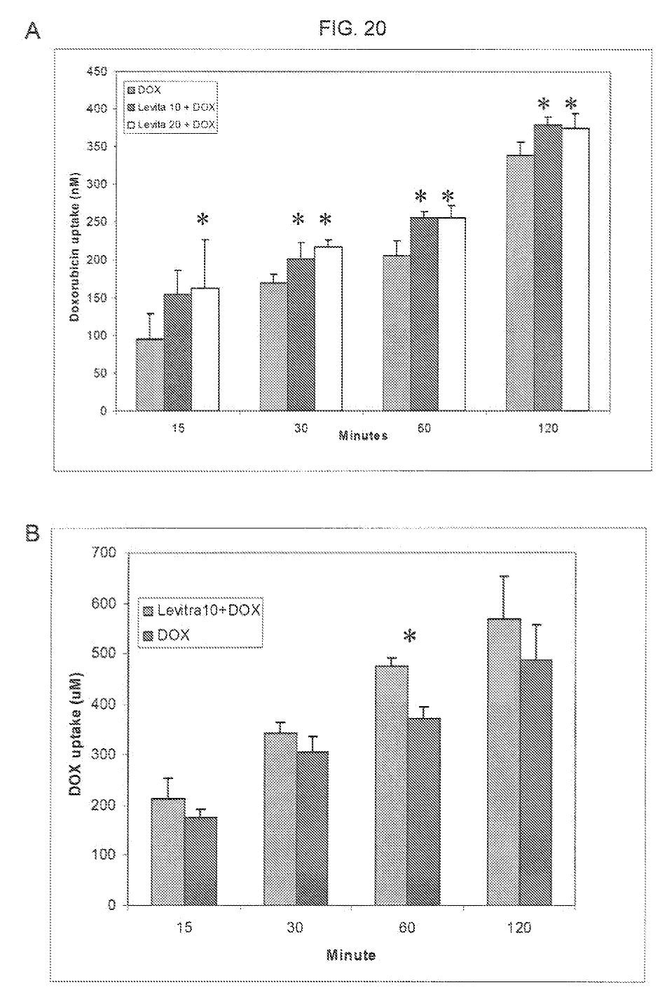

Studies further indicated that the inhibition of PDE5 enhances chemotherapeutics' transport in endothelial and/or tumor cells. ¹⁴C-sucrose was used to examine the general permeability. In these studies, the inventor also performed in vitro cellular transport studies to determine whether the inhibition of PDE5 enhances the transport of chemotherapeutics (FIG. 20; *P<0.05 compared with doxorubicin alone). Two hydrophilic chemotherapeutics, doxorubicin and carboplatin, were used. Vardenalfil significantly enhanced Adriamycin® (doxorubicin) transport in human brain endothelial cells (FIG. 20A) and CRL-5904 tumor cells (FIG. 20B).

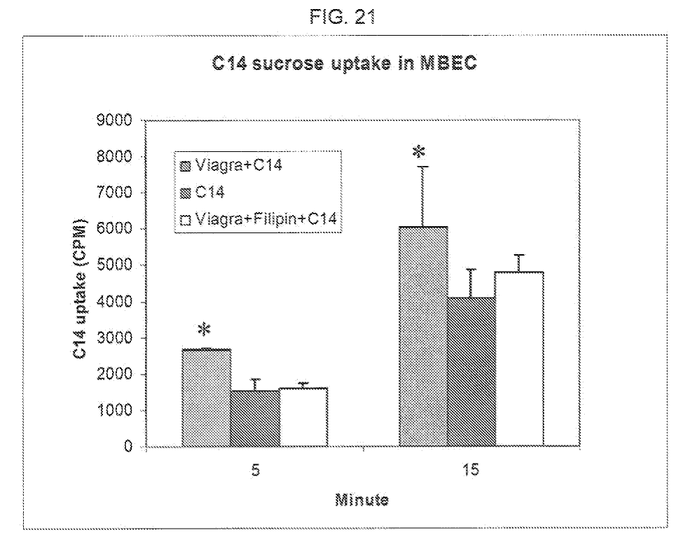

Studies further indicated that increased vesicular transport is a mechanism responsible for the increased transport by PDE5 inhibition. The inventor's previous studies suggested that vesicular transport is a mechanism for the increase of brain tumor permeability by vasoactive compounds including bradykinin and minoxidil sulfate. To determine whether this is the case for PDE5 inhibitors, the inventor used filipin, an inhibitor of caveolae endocytosis, in the in vitro cellular transport study (FIG. 21; P<0.05 vs. other two treatments). As shown, fillipin abolished the increased transport of ¹⁴C-sucrose by sildenafil. This data indicated that vesicular transport is an important mechanism responsible for the increased transport by PDE5 inhibition. As vesicular transport is not specific to brain microvessels per se, the inventor, while not wishing to be bound by any particular theory, believes the mechanism may be responsible for the increased cellular permeability by PDE5 inhibitors in non-CNS cells studied above.

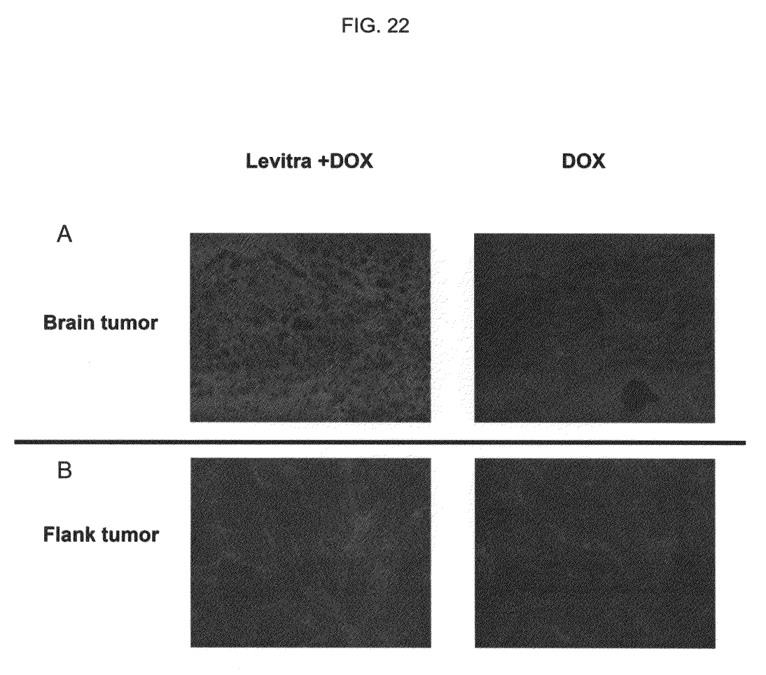

Furthermore, studies suggested that oral Levitra® (Vardenafil) increased the concentrations of the chemotherapeutic doxorubicin in brain tumor tissues as well as in flank tumor tissues of mouse models. CRL-5904 cells were implantated in nude mice. Two weeks after tumor implantation, the animals were treated with doxorubicin, with or without Levitra® (Vardenafil). Both brains and flank tumors were harvested and processed. The fluorescence of doxorubicin in tumor tissues was examined under microscopy. The preliminary data showed that Levitra® (vardenafil) treatment increased the fluorescence intensity in both brain and flank tumors (FIG. 22). However, more dramatic increase was observed for brain tumors as compared with flank tumors. The data suggest PDE5 inhibitors may have an effect on increasing the concentrations of chemotherapeutics in non-CNS tumors.

The methods of the present invention include methods of enhancing the permeability of the BTB and the abnormal BBB to a compound (e.g., therapeutic agent, imaging agent) in a subject in need thereof, comprising providing a PDE5 inhibitor or a salt thereof and administering the PDE5 or the salt thereof to the subject. The method may further include administering a therapeutic agent or an imaging agent to the subject, whereby enhanced delivery of the therapeutic agent or the imaging agent into abnormal brain tissue and/or the tumor is achieved. The selective enhancement of the permeability of the BTB and the abnormal BBB in comparison to blood vessels in normal tissue allows for the passage of the compound into the tumor or abnormal brain tissue for treatment or imaging. The tumor can be a brain tumor or a non-central nervous system tumor. The therapeutic agent or the imaging agent may be administered prior to the administration of the PDE5 inhibitor or the salt thereof, concurrently with the administration of the PDE5 inhibitor or the salt thereof or subsequent to the administration of the PDE5 inhibitor or the salt thereof, whereby the therapeutic agent or the imaging agent is delivered to the tumor during a time period of enhanced permeability. Particularly useful is to administer the therapeutic agent or imaging agent at about 30 minutes, 60 minutes, 90 minutes, 120 minutes, 150 minutes, and/or 180 minutes after the administration of the PDE5 inhibitor or the salt thereof. Although one skilled in the art will recognize that a range of times for administration may be appropriate.

The methods of the present invention also include methods of treating tumors or abnormal brain tissue in a subject in need thereof, comprising providing a PDE5 inhibitor or a salt thereof and a therapeutic agent and administering the PDE5 inhibitor or the salt thereof and the therapeutic agent to the subject. The selective enhancement of the permeability of the BTB or the abnormal BBB in comparison to capillaries in normal tissue allows for the passage or enhanced passage of the therapeutic agent into the tumor or abnormal brain tissue for treatment. The tumor can be a brain tumor or a non-central nervous system tumor. The therapeutic agent may be administered prior to the administration of the PDE5 inhibitor or the salt thereof, concurrently with the administration of the PDE5 inhibitor or the salt thereof or subsequent to the administration of the PDE5 inhibitor or the salt thereof, whereby the therapeutic agent is delivered to the tumor during a time period of enhanced permeability. Particularly useful is to administer the therapeutic agent at about 30 minutes, 60 minutes, 90 minutes, 120 minutes, 150 minutes, and/or 180 minutes after the administration of the PDE5 inhibitor or the salt thereof. Although one skilled in the art will recognize that a range of times for administration may be appropriate.

In additional embodiments, the PDE5 inhibitors and agents that increase the abnormal BBB permeability, when administered in conjunction with a therapeutic agent may treat abnormal brain tissue. During the time period that the permeability of the BBB in abnormal brain tissue is enhanced, a therapeutic agent may be administered. The selective enhanced permeability of the BBB allows passage of the therapeutic agent into the abnormal brain tissue for treatment. Examples of therapeutic agents include: anti-cancer drugs (including chemotherapeutic agents and antiproliferative agents), therapeutic viral particles, antimicrobials (e.g., antibiotics, antifungals, antivirals), mood-stabilizing agents, anticonvulsants, anti-neurodegenerative agents, anti-stroke agents, cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors).

One benefit of selective enhancement of the permeability of the abnormal BBB or BTB is that it allows for enhanced delivery of therapeutic agents or imaging agents to the abnormal brain tissue or tumors without excessively increasing the delivery of the therapeutic agents or the imaging agents to normal brain tissue or normal tissue. This may be important because the delivery of certain therapeutic agents may not be desirable for healthy tissues. For example, it is not desirable for healthy tissues/cells to be treated with chemotherapeutic agents that induce cell death.

PDE5 inhibitors and salts thereof that are useful include but are not limited to sildenafil (available under the trade name Viagra® from Pfizer, Inc.; New York, N.Y.), and vardenafil (available under the trade name Levitra® from Bayer Pharmaceuticals Corporation; Pittsburgh, Pa.). The inventor has found that oral administration of Viagra® or Levitra® causes the enhancement of the permeability of the BTB for several hours duration. Furthermore, the inventor found the dosage did not induce hypotension. This is a significant improvement over the prior art wherein the enhancement of the permeability was transient. Other PDE5 inhibitors and salts thereof will be readily recognized by those of skill in the art, and may be used in accordance with alternate embodiments of the present invention. Indeed, such other PDE5 inhibitors and salts thereof are considered to be within the scope of the present invention.

In other embodiments, a PDE5 inhibitor may be administered in combination with one or more agents that increase BTB or abnormal BBB permeability, such as bradykinin and its analogs, nitric oxide (“NO”) donor drugs, and/or potassium channel agonists. Kinins exert multiple pathophysiological functions, including vascular permeability and mitogenesis, by activating their cognate receptors, bradykinin subtype 1 receptor (B1R) and bradykinin subtype 2 receptor (B2R), which belong to the superfamily of G protein-coupled receptors.

Bradykinin, a blood hormone, increases vascular permeability, dilates blood vessels, and contracts non-vascular smooth muscle. A number of bradykinin analogs have been identified and may be used in connection with alternate embodiments of the present invention; for example, receptor mediated permeabilizer (RMP)7 or A7, [Phe⁸ ψ(CH₂—NH)Arg⁹]-bradykinin, N-acetyl-[Phe⁸ψ(CH₂—NH)Arg⁹]-bradykinin, desArg⁹-bradykinin, and related peptide structures which exhibit the same properties as bradykinin but have modified amino acids or peptide extensions on either terminal end of the peptide. See, e.g., S. R. Doctrow et al., The bradykinin analog RMP-7 increases intracellular free calcium levels in rat brain microvascular endothelial cells, J. PHARMACOL. EXP. THER., 271(1):229-237 (Oct. 1994); G. Drapeau et al., [Phe⁸ ψ(CH₂—NH)Arg⁹]bradykinin, a B2 receptor selective agonist which is not broken down by either kininase I or kininase II, EUR. J. PHARMACOL., 155:193-195 (1988); and B. M. Marcic et al., Replacement of the transmembrane anchor in angiotensin I-converting enzyme (ACE) with a glycosylphosphatidylinositol tail affects activation of the B2 bradykinin receptor by ACE inhibitors, J. BIOL. CHEM., 275:16110-16118 (2000). Additional bradykinin analogs will be readily recognized by those of skill in the art, and may be used in accordance with alternate embodiments of the present invention. Indeed, such other bradykinin analogs are considered to be within the scope of the present invention.

Similarly, a wide array of NO donor drugs may be used in accordance with various embodiments of the present invention. Examples of NO donor drugs include: organic nitrate compounds which are nitric acid esters of mono- and polyhydric alcohols, (e.g., glyceryl trinitrate (GTN) or nitroglycerin (NTG), pentaerythrityl tetranitrate (PETN), isosorbide dinitrate (ISDN), and isosorbide 5-mononitrate (IS-5-N)), S-nitrosothiol compounds (e.g., S-nitroso-N-acetyl-D,L-penicillamine (SNAP), S-nitrosoglutathione (SNOG), S-nitrosoalbumin, S-nitrosocysteine), sydnonimine compounds (e.g., molsidomine (N-ethoxycarbonyl-3-morpholino-sydnonimine), linsidomine (e.g., SIN-1; 3-morpholino-sydnonimine or 3-morpholinylsydnoneimine or 5-amino-3-morpholinyl-1,2,3-oxadiazolium), and pirsidomine (CAS 936)). Additional NO donor drugs are well known to those of skill in the art and can readily be identified and used in connection with various embodiments of the present invention.

Furthermore, potassium channel agonists may be used in connection with various embodiments of the present invention. U.S. application Ser. No. 10/938,674, entitled, “Potassium Channel Mediated Delivery of Agents through the Blood-Brain Barrier,” herein incorporated by reference in its entirety as though fully set forth, provides examples of suitable potassium channel agonists.

The PDE5 inhibitors, therapeutic agents, imaging agents and agents that increase BTB or abnormal BBB permeability (e.g., bradykinin and its analogs, NO donor drugs, potassium channel agonists) may be administered to a mammal by any conventional technique in accordance with various embodiments of the present invention to enhance BTB or abnormal BBB permeability. The PDE5 inhibitors and other enumerated agents may be delivered simultaneously or separately, by the same or different modes of administration, in therapeutically effective amounts. By way of example, a PDE5 inhibitor may be administered orally, while bradykinin may be administered intracarotidly, intraarterially, intravenously. The PDE5 inhibitors and other enumerated agents may be delivered before, concurrently with, or following administration of a therapeutic agent(s) that is targeted for enhanced delivery across the BTB or abnormal BBB.

Examples of therapeutic agents that may be used in accordance with various embodiments of the present invention include: anti-cancer drugs (including chemotherapeutic agents and antiproliferative agents), therapeutic viral particles, antimicrobials (e.g., antibiotics, antifungals, antivirals), cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors).

Examples of chemotherapeutic agents include cytotoxic agents (e.g., 5-fluorouracil, cisplatin, carboplatin, methotrexate, daunorubicin, doxorubicin (Adriamycin®), vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Examples of antiproliferative agents include alkylating agents, antimetabolites, enzymes, biological response modifiers, hormones and antagonists, androgen inhibitors (e.g., flutamide and leuprolide acetate), antiestrogens (e.g., tamoxifen citrate and analogs thereof, toremifene, droloxifene and roloxifene), Additional examples of antiproliferative agents include, but are not limited to levamisole, gallium nitrate, granisetron, sargramostim strontium-89 chloride, filgrastim, pilocarpine, dexrazoxane, and ondansetron.

Anti-neurodegenerative agents include, for example, anticholinergics, dopamine precursors (e.g., L-dopa (Sinemet, carbidopa)), COMT inhibitors, dopamine receptor agonists, MAO-B inhibitors, bromocriptine (Parlodel), pergolide (Permax), benztropine (Cogentin), amantadine (Symmetrel), trihexyphenidyl (Artane) and deprenyl (Eldepryl, selegiline), Huperzine A, acetylcholinesterase (AChE) inhibitors, N-methyl-D-aspartate (NMDA) receptor antagonists (e.g., Namenda (Memantine)), and cholinesterase inhibitors (e.g., Aricept (donepezil), Reminyl (Galantamine), Exelon (rivastigmine), Cognex (Tacrine).

Additionally, U.S. application Ser. No. 10/938,674, entitled, “Potassium Channel Mediated Delivery of Agents through the Blood-Brain Barrier,” herein incorporated by reference in its entirety as though fully set forth, provides further information on suitable therapeutic agents. Other suitable therapeutic agents will also be readily recognized by those of skill in the art.

In various embodiments, the present invention provides pharmaceutical compositions including a pharmaceutically acceptable excipient along with a therapeutically effective amount of a PDE5 inhibitor or a salt thereof; for example sildenafil, vardenafil, salts thereof, alone or in combination. In a further embodiment, the pharmaceutical composition may further comprise a therapeutic agent. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluents, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Dosages may also be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied abnormal tissue, or the responses observed in the appropriate animal models, as previously described. In the rat model, typical dosages of a PDE5 inhibitor or salt thereof may be from about 10 to about 50 mg/kg. In particular embodiments, dosages of sildenafil in human subjects may be about 10 mg to about 200 mg, about 25 mg to about 100 mg, and more particularly about 25 mg, 50 mg, 100 mg, or 200 mg. In other particular embodiments, dosages of vardenafil in human subjects may be about 1 mg to about 40 mg, about 2.5 mg to about 20 mg, and more particularly, about 2.5 mg, 5 mg, 10 mg, 20 mg, or 40 mg. It may be beneficial to administer the PDE5 inhibitor or salt thereof about 30 to about 45 minutes or about 30 to about 60 minutes prior to administering a therapeutic agent or imaging agent. In various embodiments, it may be beneficial to administer the PDE5 inhibitors about 30 minutes to about 180 minutes prior to the administration of the therapeutic agent or the imaging agent. In particular embodiment, it may be beneficial to administer the PDE5 inhibitor or salt thereof about PDE5 inhibitors about 30 minutes, 60 minutes, 90 minutes, 120 minutes, 150 minutes or 180 minutes prior to the administration of the therapeutic agent or the imaging agent.

The present invention is also directed to a kit for selectively enhancing the permeability of the BTB or the abnormal BBB or to treat a tumor. The kit is useful for practicing the inventive method of selectively enhancing the permeability of the BTB or to treat a tumor. The kit is an assemblage of materials or components, including at least one of the inventive compositions. Thus, in some embodiments the kit contains a PDE5 inhibitor; for example, sildenafil, vardenafil or a salt thereof. In other embodiments, the kit may further include a therapeutic agent or an imaging agent.

The exact nature of the components configured in the inventive kit depends on its intended purpose. For example, some embodiments are configured for the purpose of selectively enhancing the permeability of the BTB or the abnormal BBB. Other embodiments are configured for the purpose of treating tumors. Still other embodiments are configured for the purpose of imaging a tumor. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as selectively enhancing the permeability of the BTB or the abnormal BBB. Instructions for use may include, for example, instructions to administer a PDE5 inhibitor in an amount sufficient to selectively enhance the permeability of the BTB or the abnormal BBB, instructions to administer a PDE5 inhibitor prior to the administration of a therapeutic agent, instructions to administer a PDE5 inhibitor about 30 to about 45 minutes or about 30 to about 60 minutes prior to the administration of a therapeutic agent, and/or instruction to administer a PDE5 inhibitor via oral, intravenous, intraarterial, or intracarotid administration and/or instructions to administer an additional agent that enhances the permeability of the BTB or the abnormal BBB (e.g., bradykinin, nitrogen oxide donor drug, potassium channel agonist, salts thereof, and analogs thereof). Optionally, the kit also contains other useful components, such as, bradykinins, analogs or salts thereof, NO donor drugs, additional potassium channel agonists, therapeutic agents, imaging agents diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in treating tumors. As used herein, the term “package” refers to a suitable solid matrix or material such as glass, plastic, paper, foil, and the like, capable of holding the individual kit components. Thus, for example, a package can be a bottle used to contain suitable quantities of a PDE5 inhibitor or a salt thereof. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Brain Permeability Determination

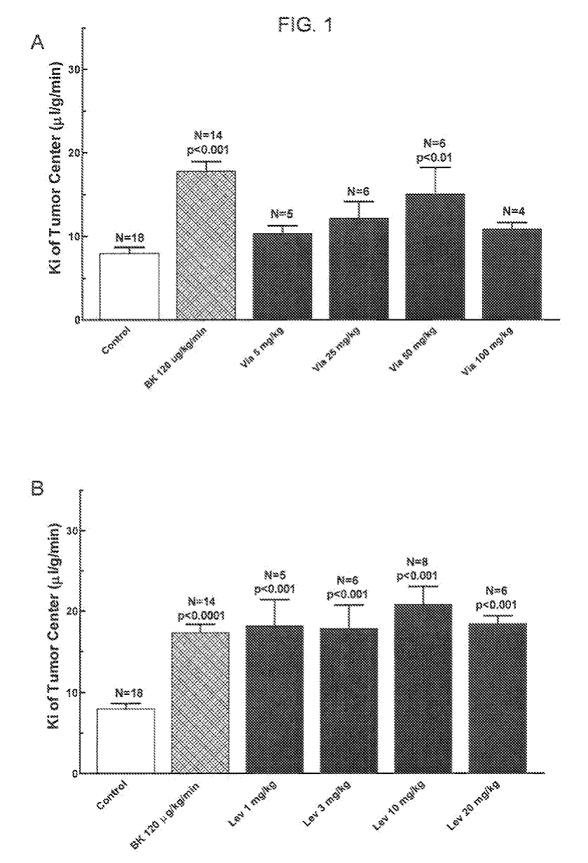

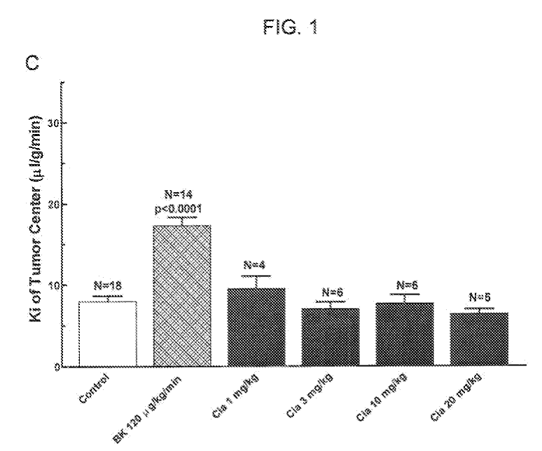

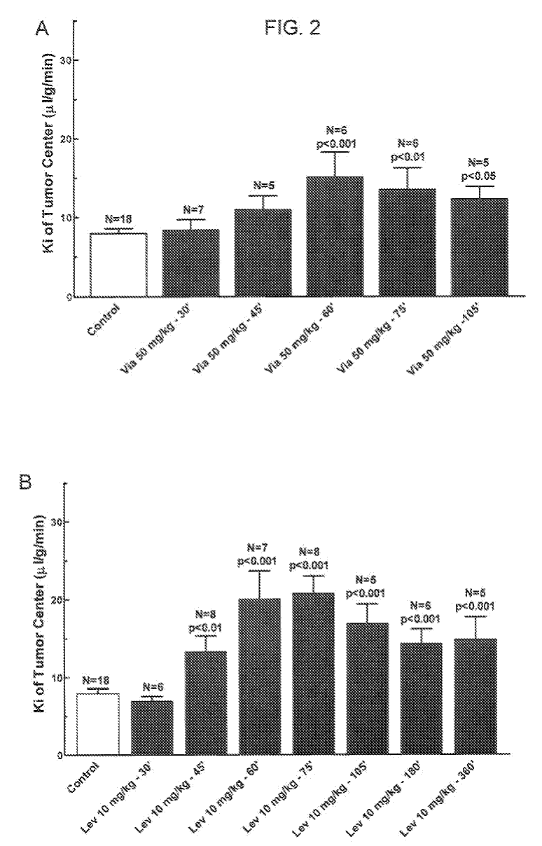

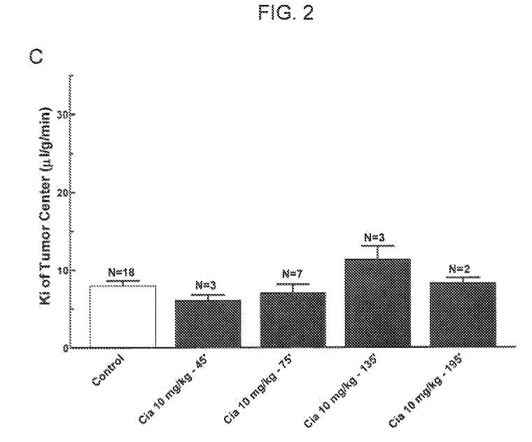

Adult, female Fischer rats (180-200 g) were implanted with 9L glioma cells (100,000 cells). Six days after tumor implantation, a PDE5 inhibitor (Viagra® (Sildenafil), Levitra® (Vardenafil) or Clalis® (Tadalafil)) is given orally 45-60 min before the permeability (“Ki”) determination. Permeability determinations were performed. See FIGS. 1 and 2.

### Example 2

### Catheter Cannulation

Polyethylene catheters were inserted into the femoral vessels. A single venous catheter was implanted for the administration of saline (control) or bradykinin (“BK”) and [¹⁴C]-sucrose radiotracer. One femoral arterial catheter was implanted for the collection of blood and another for the monitoring of blood pressure. Catheters were secured with silk sutures and flushed with heparinized saline.

### Example 3

### Drug Administration

Subjects received either BK or saline, infused at a rate of 66.7 μl/min over 15 minutes. A small sample of arterial blood was analyzed for hematocrit, pO₂, pCO₂, Na⁺, K⁺ and pH. Baseline blood pressure was measured for one minute, then i.v. saline or drug infusion was initiated. Five minutes later, [¹⁴C]-sucrose (10 μCi i.v., in a 0.2 ml bolus) was injected and continuous arterial blood withdrawal was initiated at a rate of 0.083 ml/min.

### Example 4

### Blood & Tissue Processing

After 15 minutes of drug administration, continuous blood collection was terminated and 0.5 ml of “stop blood” was collected. Replicate 20 μl aliquots from continuous and stop blood were mixed with 250 μl tissue solubilizer, then with 500 μl 30% hydrogen peroxide, and incubated overnight. Thereafter, they were analyzed for [¹⁴C] using liquid scintillation counting. Subjects were decapitated and their brains removed and snap-frozen on dry ice, then stored at −20° C. See FIGS. 1 and 2.

### Example 5

### Effect of Combining PDE5 inhibitor and Bradykinin on Tumor Permeability

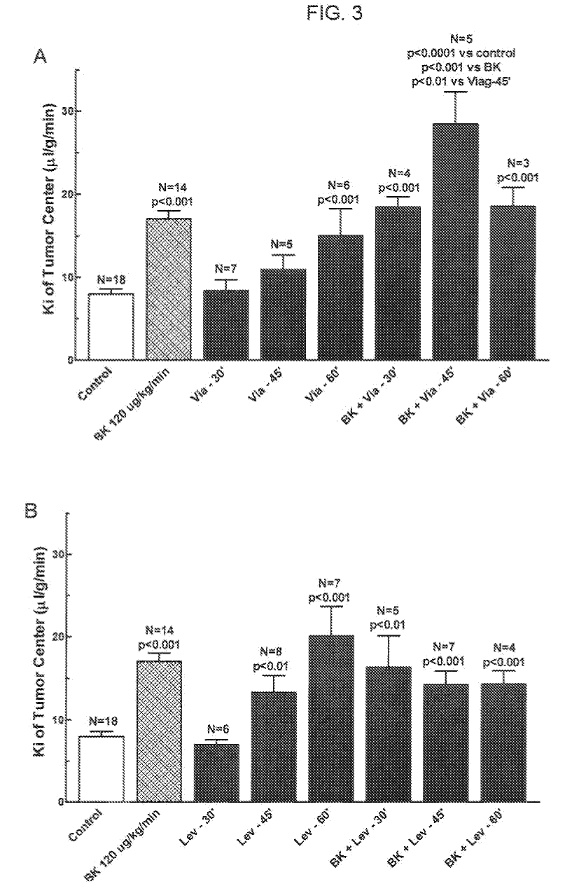

PDE5 inhibitors (Viagra® (Sildenafil) 50 mg/kg and Levitra® (Vardenafil) 10 mg/kg) were administered orally followed by bradykinin infusion for the final 15 minutes. See FIG. 3.

### Example 6

### Quantitative Autoradiography (QAR)

Coronal brain sections were exposed to a phosphor screen (5 days) and scanned. Densitometry data were sampled from 6 brain sections containing the largest tumor area in the following regions: tumor core, brain surrounding tumor, neocortex, white matter and basal ganglia. Densitometry data were used to determine the rate of blood-to-brain transfer of [¹⁴C]-sucrose (Ki, in μl/g/min).

### Example 7

### Survival Studies

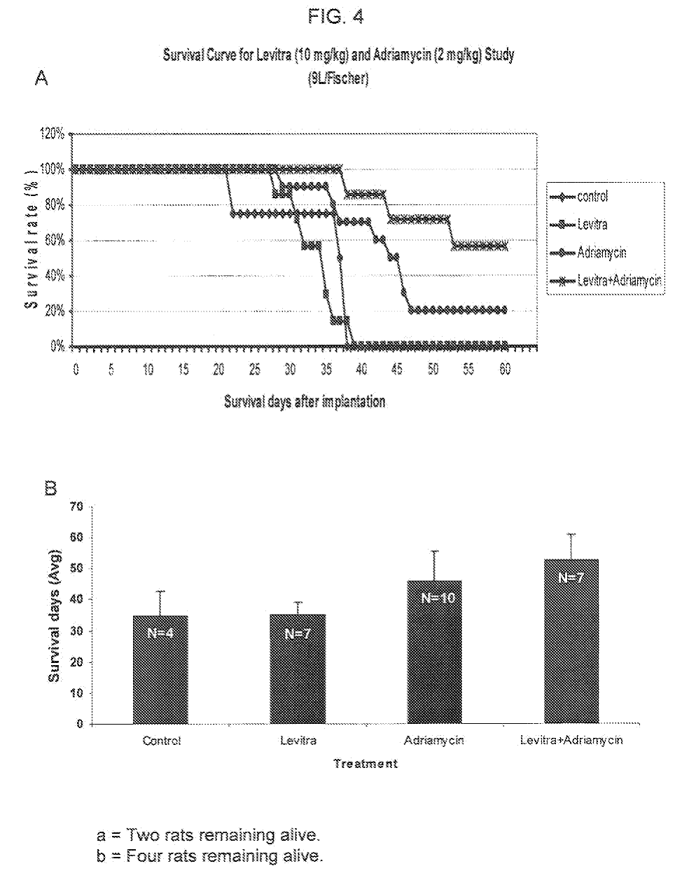

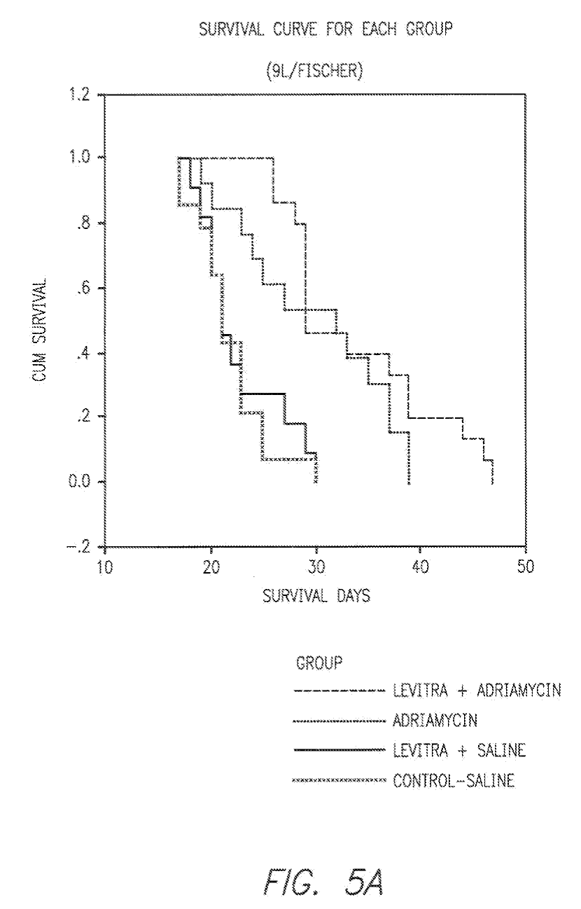

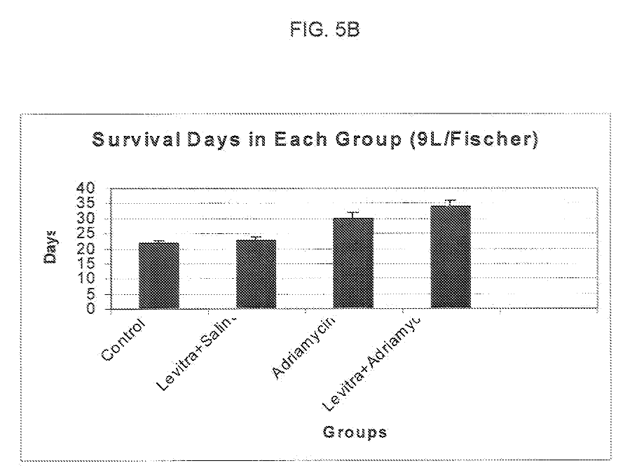

Adult, female Fischer rats (180-200 g) were implanted with 9L glioma cells (100,000 cells). For three consecutive days starting either 4 or 7 days following tumor implantation, animals were treated with Adriamycin® (doxorubicin) (2 mg/kg i.v. tail-vein injection) after 45 minutes following oral administration of Levitra®) (vardenafil) (10 mg/kg). See FIGS. 4 and 5. The study on the treatment on days 4, 5 & 6 post tumor implantation was in progress and the data show in FIG. 4 represents the data available as of Feb. 15, 2006. Table 1 depicts the log rank statistical analysis of the effect of Levitra® (vardenafil) and Adriamycin® (doxorubicin) treatment on days 7, 8 & 9 post tumor implantation.

TABLE 1

| Mean Survival Days | N | Mean | SD |
|---|---|---|---|
| Control | 14 | 21.8 | 0.9 |
| Levitra ® (vardenafil) + Saline | 11 | 22.7 | 1.2 |
| Adriamycin ® (doxorubicin) | 13 | 30 | 2.0 |
| Levitra ® (vardenafil) + Adriamycin ® (doxorubicin) | 15 | 34 | 1.9 |

Log Rank Statistical Analysis
Control vs Adriamycin ® (doxorubicin): p < 0.05;
Control vs Levitra ® (vardenafil) + Adriamycin ® (doxorubicin): p < 0.05;
Adriamycin ® (doxorubicin) vs Levitra ® (vardenafil) + Adriamycin ® (doxorubicin): p = 0.1255;
Control vs Levitra ® (vardenafil) + Saline: p = 0.7357

### Example 8

### Survival Rate

Rats were monitored daily to determine survival times of the tumor-bearing rats for either death or when animals became moribund, euthanasia was employed. See FIGS. 4 and 5.

### Example 9

### cGMP Determination

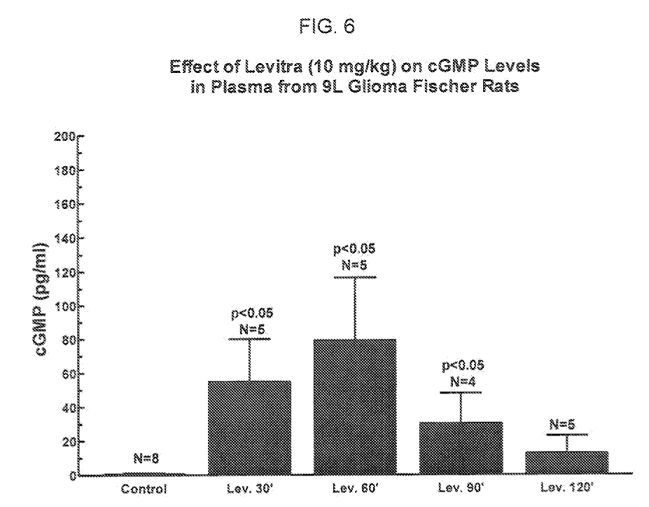

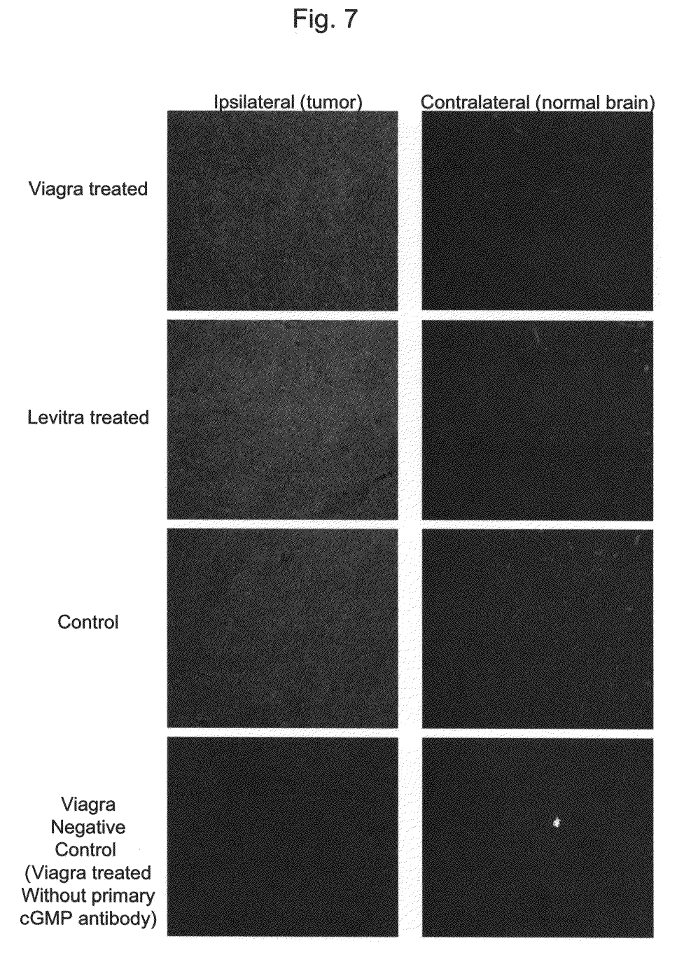

cGMP levels were determined using a ELISA-based kit (Cayman Chemical Co., Ann Arbor, Mich.) for plasma and immunohistocytochemistry with anti-cGMP antibody (Chemicon Corp.) for tissue sections. See FIGS. 6 and 7.

### Example 10

### Administration of a PDE5 Inhibitor for Enhanced Permeability for a Therapeutic Agent

A PDE5 inhibitor is administered orally to a mammal prior to administration of a therapeutic agent for the treatment of a brain disease. The PDE5 inhibitor is administered from about 30 minutes to about 45 minutes prior to administration of the therapeutic agent. Administration of the PDE5 inhibitor markedly enhances the permeability of the BBB with respect to the therapeutic agent; thereby allowing for effective treatment of the brain disease.

### Example 11

### Administration of Sildenafil for Enhanced Permeability for a Brain Tumor Therapeutic Agent

Sildenafil is administered orally to a mammal prior to administration of an anti-cancer drug for the treatment of a brain tumor. The sildenafil is administered from about 30 minutes to about 45 minutes prior to administration of the anti-cancer drug; for example Adriamycin® (doxorubicin). Administration of sildenafil markedly enhances the permeability of the BBB with respect to the anti-cancer drug; thereby allowing for effective treatment of the brain tumor.

### Example 12

### Administration of Vardenafil for Enhanced Permeability for a Brain Tumor Therapeutic Agent

Vardenafil is administered orally to a mammal prior to administration of an anti-cancer drug for the treatment of a brain tumor. The vardenafil is administered from about 30 minutes to about 45 minutes prior to administration of the anti-cancer drug; for example Adriamycin® (doxorubicin). Administration of vardenafil markedly enhances the permeability of the BBB with respect to the anti-cancer drug; thereby allowing for effective treatment of the brain tumor.

### Example 13

### Administration of Sildenafil and Bradykinin for Enhanced Permeability for a Brain Tumor Therapeutic Agent

Sildenafil is administered orally to a mammal from about 30 minutes to about 45 minutes prior to administration of an anti-cancer drug for the treatment of a brain tumor. A quantity of bradykinin is administered to the mammal via intravenous, intraarterial, and/or intracarotid infusion about 15 minutes prior to the administration of the anti-cancer drug for the treatment of a brain tumor. Administration of sildenafil and bradykinin markedly enhances the permeability of the BBB with respect to the anti-cancer drug; thereby allowing for effective treatment of the brain tumor.

### Example 14

### Administration of a PDE5 inhibitor and a Therapeutic Agent to Treat Abnormal Brain Tissue

A therapeutically effective amount of a PDE5 inhibitor is administered orally to a mammal from about 30 minutes to about 45 minutes prior to administration of a therapeutic agent. Thereafter, a therapeutic agent is administered to the mammal via oral, intravenous, intraarterial, and/or intracarotid administration. The administration of the PDE5 inhibitor markedly enhances the permeability of the BBB with respect to the therapeutic agent; thereby allowing for effective treatment of the abnormal brain tissue.

### Example 15

### Administration of Sildenafil for Enhanced Permeability for Treatment with an Anti-Neurodegenerative Agent

Sildenafil is administered orally from about 30 minutes to about 45 minutes prior to administration of an anti-neurodegenerative agent to treat the neurodegenerative disease. Administration of sildenafil markedly enhances the permeability of the BBB with respect to the anti-neurodegenerative agent; thereby allowing for effective treatment of the neurodegenerative disease.

### Example 16

### Animals and Materials

All animal experiments were conducted in accordance with policies set by the Institutional Animal Care and Use Committee at Cedars-Sinai Medical Center and by NIH guidelines. Female Fischer rats, weighting 150-180 g, were used for this study. Bradykinin (BK) was obtained from the Sigma Co. (St. Louis, Mo.), sildenafil (Viagra®) from Pfizer, Inc (New York, N.Y.), vardenafil (Levitra®) from the Bayer Pharmaceuticals Co. (West Haven, Conn.), and iberiotoxin from Sigma (Natik, Mass.). Adriamycin® (doxorubicin hydrochloride) was obtained from Ben Venue Laboratories, Inc. (Bedford, Ohio). [¹⁴C]-Sucrose (363 mCi/mmol) was obtained from ICN Biomedicals, Inc. (Dupont New England Nuclear, Boston, Mass.).

### Example 17

### Intracerebral Tumor Implantation

9L gliosarcoma cells were kept frozen until use, and then thawed and maintained in a monolayer culture in DMEM medium with 10% FBS. The rats were anesthetized with intraperitoneal injections of ketamine (50 mg/kg)/xylazine (6 mg/kg), and immobilized in a stereotactic frame. The implantation (1×10⁵ 9L glioma cells) was conducted as previously described (Ningaraj et al., Regulation of blood-brain tumor barrier permeability by calcium-activated potassium channels. J PHARMACOLOGY AND EXPERIMENTAL THERAPEUTICS. 2002; 301: 838-851; Sugita et al., Cyclic GMP-specific phosphodiesterase inhibition and intracarotid bradykinin infusion enhance permeability into brain tumors. CANCER RES. 1998; 58: 914-920).

### Example 18

### Animal Preparation

For Ki study described below, six days after tumor implantation, the rats were anesthetized with ketamine/xylazine. One femoral vein was cannulated for administration of the appropriate drug and the radiotracer; one femoral artery was cannulated to withdraw arterial blood, and another femoral artery was cannulated to monitor systemic blood pressure. Body temperature was maintained at 37° C., and arterial blood gases, blood pressure and hematocrit were monitored. Animals that showed physiologic parameters outside a predetermined range during the procedures were eliminated from the study. The rats were treated with: (1) intravenous (i.v.) infusion of saline; (2) intravenous infusion of BK (120 μg/kg/min); (3) oral sildenafil; (4) oral vardenafil group; (5) oral sildenafil+i.v. BK; (6) oral vardenafil+i.v. BK; or (7) oral vardenafil+iberiotoxin (0.26 μg/kg/min).

### Example 19

### [¹⁴C] Sucrose Transport Studies

The method used to determine Ki, initial transport of a radioactive tracer from blood into tissue, has been described in the inventor's previous publications (Matsukado et al., Enhanced tumor uptake of carboplatin and survival in glioma-bearing rats by intracarotid infusion of bradykin analog, RMP-7. NEUROSURGERY. 1996; 39: 125-134; 13; Ningaraj et al., Regulation of blood-brain tumor barrier permeability by calcium-activated potassium channels. J PHARMACOLOGY AND EXPERIMENTAL THERAPEUTICS. 2002; 301: 838-851; 25; Ningaraj et al., Adenosine 5′-triphosphate-sensitive potassium channel-mediated blood-brain tumor barrier permeability increase in a rat brain tumor model. CANCER RES. 2003; 63: 8899-88911) with minor modifications. In brief, [¹⁴C] sucrose was used as the radiotracer. Either BK or saline was infused into the femoral vein at a rate of 66.7 μl/min for 15 minutes. For rats treated with oral PDE 5 inhibitors, the dose specified below was administrated by oral gavage. Five minutes (ten minutes before decapitation) after the start of the intravenous infusion, or in the case of oral PDE 5 inhibitors 10 minutes before decapitation, 50 μCi/kg of [¹⁴C] sucrose was injected as an intravenous bolus. Serum radioactivity was determined for 10 minutes after tracer injection. After completion of the experiments, the animals were euthanized by decapitation and the brains immediately removed and frozen.

To determine the dose response for the Ki, various doses of sildenafil (5, 25, 50 and 100 mg/kg) or vardenafil (1, 3, 10 and 20 mg/kg) were administered. The maximal effects on permeability by sildenafil and vardenafil were achieved at 50 mg/kg and 10 mg/kg, respectively, which were then employed for subsequent experiments. To study the duration of increased permeability induced by sildenafil or vardenafil, the Ki was determined at different time points from 30 to 105 minutes after oral administration of 50 mg/kg sildenafil or 10 mg/kg vardenafil.

### Example 20

### Quantitative Autoradiography and Ki Calculation

The frozen brains were mounted onto pedestals with Ml embedding matrix, after which 20 μm coronal sections were cut with a cryostat. The sections were thaw-mounted onto slides, and autoradiographs were generated by exposing the sections along tissue-calibrated ¹⁴C standards on a phosphor screen for 5 days. Quantitative analysis of the regional radioactivity for tumor and other brain areas was performed using a computer (Power Macintosh 7100) and Image 1.55 software (National Institutes of Health, Bethesda, Md.). The initial rate for blood-to-brain transfer (Ki) was calculated as previously described (Nomura et al., Intracarotid infusion of bradykinin selectively increases blood-tumor permeability in 9L and C6 brain tumors. BRAIN RES. 1994; 659: 62-66; Black et al., Intracarortid infusion of RMP-7, a bradylinin analog, increases transport of gallium-68 EDTA into human glioma. J NEUROSURG. 1997; 86: 603-609; Ningaraj et al., Regulation of blood-brain tumor barrier permeability by calcium-activated potassium channels. J PHARMACOLOGY AND EXPERIMENTAL THERAPEUTICS. 2002; 301: 838-851; Ningaraj et al., Adenosine 5′-triphosphate-sensitive potassium channel-mediated blood-brain tumor barrier permeability increase in a rat brain tumor model. CANCER RES. 2003; 63: 8899-88911).

### Example 21

### Plasma cGMP Assay

Intracerebral inoculations of 9L tumor cells were performed as described above. On day 6 after tumor implantation, rats were treated with 10 mg/kg vardenafil by gavage. Different groups of rats were anesthetized 5 minutes before and at 30, 60, 90 and 120 minutes after treatment respectively and the femoral vein was cannulated. Blood was collected and the plasma was separated. The cGMP concentration in the plasma was determined using a commercially available ¹²⁵I-labeled cGMP assay kit (Amersham, Arlington Heights, Ill.). cGMP content was expressed as pmol/ml of plasma.

### Example 22

### Brain cGMP Immunohistochemistry

Rats with a 9L tumor in the brain were decapitated before and at 30, 60, 90 and 120 minutes after vardenafil treatment. The procedures for immunohistochemistry was previously describe (Ningaraj et al., Regulation of blood-brain tumor barrier permeability by calcium-activated potassium channels. J PHARMACOLOGY AND EXPERIMENTAL THERAPEUTICS. 2002; 301: 838-851) except that cGMP antibody was used in this study. Tissue sections were incubated with rabbit anti-cGMP diluted in 1:1000 in PBS. Localization of the cGMP antibody was accomplished by incubation of sections for 1 hour at room temperature with anti-rabbit IgG conjugated with fluorescent. The images were obtained by Zeiss fluorescece microscopy (Carl Zeiss MicroImaging, Inc., Thornwood, N.Y.). The immunofluoresence intensity of cGMP staining was measured in 4 randomly chosen areas within the tumor, in the brain surrounding the tumor (BST; within 2 mm of the border of the tumor), and in contralateral cortex using Zeiss AxionVision software. Pictures of the tumor tissue were taken with a magnification of 20× under the same conditions.

### Example 23

### Transmission Electron Microscopy (TEM)

TEM studies were performed as previously described with modifications (Ningaraj et al., Regulation of blood-brain tumor barrier permeability by calcium-activated potassium channels. J PHARMACOLOGY AND EXPERIMENTAL THERAPEUTICS. 2002; 301: 838-851; Hashizume K and Black K L., Increased endothelial vesicular transport correlated with increased blood-tumor barrier permeability induced by bradykinin and leukotrienes C4. J NEUROPATHOL EXP NEUROL 2002; 61: 725-735). In brief, tumor implantation and animal preparation were conducted as described above. The rats were treated with vardenafil (10 mg/kg, p.o.) or PBS (n=4) and sacrificed one (n=3), two (n=4), or three (n=4) hours later. An additional group of rats (n=3) was intravenously infused with BK (10 μg/kg/min in PBS) for 10 minutes. Five minutes before sacrifice, the rats were intravenously given a bolus of horse radish peroxidase (HRP) (200 mg/kg) which is the enzymatic tracer for transcytosis of blood borne protein through the BBB. (Hashizume et al., Increased endothelial vesicular transport correlated with increased blood-tumor barrier permeability induced by bradykinin and leukotrienes C4. J NEUROPATHOL EXP NEUROL 2002; 61: 725-735; Nishio et al., Peripheral nerve lesions associated with cis-diamminedichloroplatinum (II) treatment-study of a case with yolk sac tumor of the pineal body. RINSHO SHINKEIGAKU 1983; 23: 655-660; Broadwell et al., Angioarchitecture of the CNS, pituitary gland, and intracerebral grafts revealed with peroxidase cytochemistry. J COMP NEUROL 1987; 260: 47-62). Ten minutes after HRP injection, rats were perfuse-fixed and the brains were removed and sectioned with a Leica VT100S vibrating blade microtome (40 μm sections). The sections were incubated to develop the substrate for HRP and then postfixed with 1% osmium tetroxide and 1% potassium ferracyanide. After dehydrated in ascending ethanol series and propylene oxide, the tissue was infiltrated with a mixture of resin and propylene oxide (3:1) and then with 100% resin. Epon embedded sections were examined on a microscope. Selected areas were cut and mounted on an epon block for thin sectioning. The thin sections were cut with an ultramicrotome to a thickness of ˜70 nm which was examined on a JEOL 1101 electron microscope (JEOL, Tokyo, Japan).

### Example 24

### Analysis of Vesicles and Tight Junction Integrity

The criteria of selecting microvessels, vesicles and tight junctions for quantitative analysis was reported as previously described (Hashizume et al., Increased endothelial vesicular transport correlated with increased blood-tumor barrier permeability induced by bradykinin and leukotrienes C4. J NEUROPATHOL EXP NEUROL 2002; 61: 725-735; Stewart et al., Quantitative study of microvessel ultrastructural in human peritumoral brain tissue. Evidence for a blood-brain barrier defect. J NEUROSURG 1987; 67: 697-705). Ten to fifteen profiles of capillaries sectioned transversely in each group were selected and photographed at low magnification (×6,000) for evaluation of their general features. Then four test zones of each endothelial cytoplasm were photographed at higher magnification (×60,000). Three to five vessels were sampled from each rat and each animal group consisted of 15-20 capillaries and a population of 60-80 test zones. Each test zone was analyzed for vesicle density (40-70 nm size of vesicle) and tight junction integrity using TEM Imaging Platform iTEM from Olympus soft Imaging System.

### Example 25

### Real-Time PCR

Total cellular RNA was extracted from 9L tumor cells using TRIzol Reagent (Invitrogen, Carlsbad, Calif.) according to the manufacture's protocol. One microgram (1 μg) total RNA was reverse-transcribed into cDNA using iScript™ cDNA Synthesis kit (Bio-Rad Laboratories, Hercules, Calif.). Targeting gene primers (PDE1b, PPR50677A; PDE5a, PPR45092A; PDE10a, PPR49833A.) were purchased from SuperArray (Frederick, Md.). GAPDH is used as the internal control. Dural-color, real time quantitative RT-PCR was performed using SYBR Green method (Bio-Rad Laboratories, Hercules, Calif.). The mRNA expression level was normalized by GAPDH expression.

### Example 26

### Survival Study

For the survival experiments, implantation of 9L gliosarcoma cells (9L/Luc), (Rehemtulla et al., Rapid and quantitative assessment of cancer treatment response using in vivo bioluminescence imaging. NEOPLASIA. 2000; 2: 491-495) which stably express luciferase, was performed using the same method as described above. Tumor-bearing rats were randomly divided into four groups as follows: (1) Control, saline-treated (N=8); (2) vardenafil-treated (10 mg/kg, orally, N=7); (3) Adriamycin® (doxorubicin)-treated (2 mg/kg, intravenously, N=8); and (4) vardenafil (10 mg/kg, orally)+Adriamycin® (doxorubicin) group (2 mg/kg, intravenously) (N=6). The rats received their treatments for three consecutive days beginning at day 4 after tumor implantation. In Ki studies, vardenafil was found to be more effective in increasing Ki than sildenafil, so vardenafil was used in survival studies. Based on the Ki studies, 10 mg/kg of vardenafil was chosen and administrated by gavage 45 minutes before Adriamycin® (doxorubicin) treatment. The dose of Adriamycin® (doxorubicin) (2 mg/kg, injected into tail vein) was based on the inventor's data and a previous publication (Wolff et al., Chemosensitivity of glioma cells in vitro: a meta analysis. J CANCER RES CLIN ONCOL. 1999; 125:481-486). The rats were monitored carefully for clinical signs attributable to brain tumor growth or until death. The efficacy of therapy was estimated by the median survival time of the animals.

### Example 27

### Tumor Size Monitoring

The noninvasive bioluminescence imaging technology, Xenogen IVIS200 Image System (Xenogen Corporation, Alameda, Calif.), was used to monitor the antitumor effects of vardenafil and/or Adriamycin® (doxorubicin) in the survival studies (Mendel et al., In vivo antitumor activity of SU11248, a novel tyrosine kinase inhibitor targeting vascular endothelial growth factor and platelet derived growth factor receptors: Determination of a pharmacokinetic/pharmacodynamic relation. CLINICAL CANCER RES. 2003; 9: 327-337). After implantation of 9L cells that express luciferase,30 tumors were allowed to grow untreated for 4 days, at which time the rats underwent initial imaging. The animals were given an aqueous solution of D-luciferin (150 mg/kg, administered intraperitoneally) 18 minutes prior to imaging. This time point was based on the results from the inventor's pilot studies.

The rats were also imaged at day 9 after drug treatment. Data were analyzed using the Xenogen LivingImage software. In a separate experiment, other four groups of rats were given the same treatments and sacrificed at day 9 after the last treatment. The frozen brains were mounted and cut with a cryostat. The sections were then stained with hematoxylin and eosin (H and E) for histological examination.

### Example 28

### Statistical Analysis

Results are expressed as mean±standard error (SEM), where applicable. The statistical analyses of Ki, cGMP levels, vesicle numbers and survival time between different groups, with or without the drug treatment, were performed using ANOVA, followed by either unpaired Student's t test of parametric analysis or by the Mann-Whitney U test of nonparametric analysis. Kaplan-Meier plot was used to analyze survival. A p-value of less than 0.05 was considered statistically significant.

### Example 29

### Expression of PDE1, PDE5 and PDE10 in 9L Tumor Cells

First examined, by using real-time PCR, mRNA levels of PDEs in 9L, was a gliosarcoma cell line that was used to generate the brain tumor model in this study. mRNA of three PDEs, PDE1, PDE5 and PDE10 were detected. However, mRNA levels of PDE5 were much higher than those of PDE1 and PDE10 (FIG. 8). PDE5 mRNA was also highly detected in other brain tumor cell lines such as GL26, U87, RG2, and importantly, a human microvessel endothelial cell line as well as human brain tumor samples available in the inventor's laboratory (data not shown).

### Example 30

### Effects of Sildenafil and Vardenafil on BTB Permeability (Ki)

It was previously reported a high correlation between the initial transport, Ki, of [¹⁴C] sucrose and the initial transport of water soluble chemotherapeutic agents including [¹⁴C]carboplatin (Ningaraj et al., Adenosine 5′-triphosphate-sensitive potassium channel-mediated blood-brain tumor barrier permeability increase in a rat brain tumor model. CANCER RES. 2003; 63: 8899-88911) and [¹¹C] methotrexate (Inamura et al., Intracarotid histamine infusion increases blood tumor permeability in RG2 glioma. NEUROL RES. 1994; 16: 125-128) in rat brain tumor models. [¹⁴C] sucrose was therefore used in this study to examine the BTB permeability as reported before (Matsukado et al., Enhanced tumor uptake of carboplatin and survival in glioma-bearing rats by intracarotid infusion of bradykin analog, RMP-7. NEUROSURGERY. 1996; 39: 125-134; 25; Ningaraj et al., Adenosine 5′-triphosphate-sensitive potassium channel-mediated blood-brain tumor barrier permeability increase in a rat brain tumor model. CANCER RES. 2003; 63: 8899-88911.) The effects of two marketed PDE5 inhibitors, sildenafil, and vardenafil on the initial blood to brain or blood to tumor transport, Ki, was studied and compared with that of BK, which increases BTB transport (Nomura et al., Intracarotid infusion of bradykinin selectively increases blood-tumor permeability in 9L C6 brain tumors. BRAIN RES. 1994; 659: 62-66; Inamura et al., Bradykinin selectively opens blood-tumor barrier in experimental brain tumors. J CEREB BLOOD FLOW METAB. 1994; 14: 862-870).

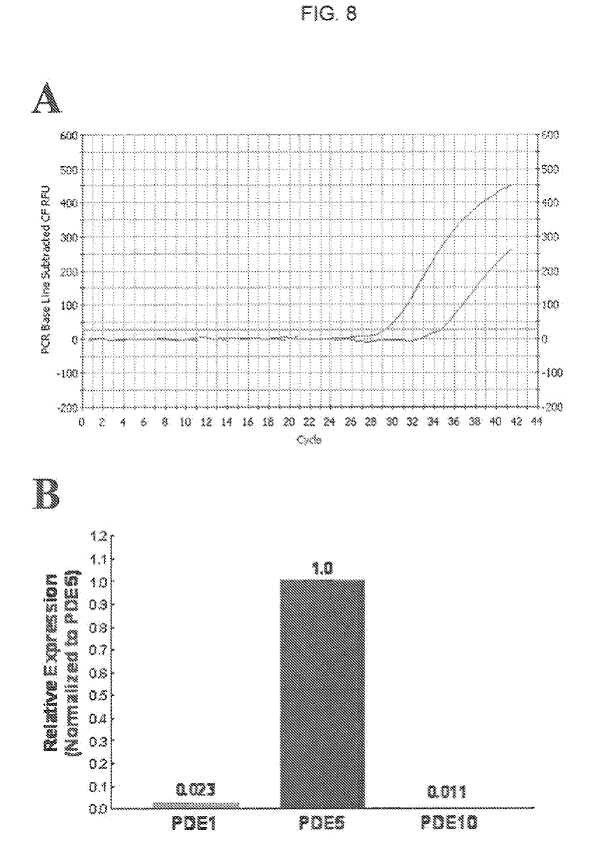

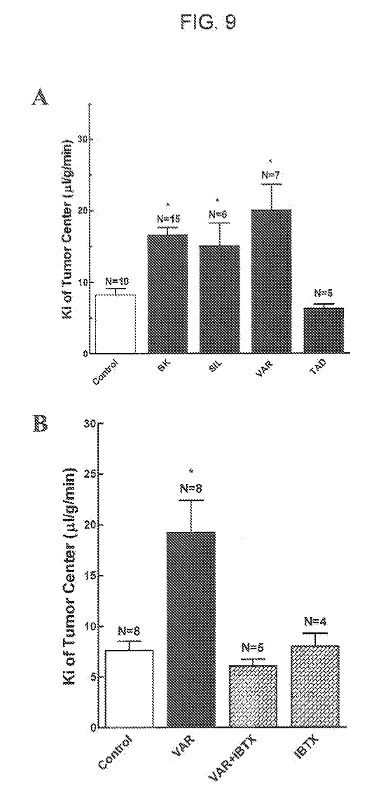

Consistent with previous reports, intravenous infusion of BK (120 μg/kg/min for 15 minutes) significantly increased the Ki (16.56±1.03 μl/g/min versus 8.26±0.89, p<0.001) as compared to the untreated controls. The Ki was also significantly increased in the rats treated with 50 mg/kg sildenafil (15.03±3.21 μl/g/min, p<0.01 versus the control), and vardenafil significantly increased the Ki at various doses with the maximal effect at 10 mg/kg (20.05±3.59 μl/g/min, p<0.001 versus the control) (FIG. 9A). BK resulted in a 2.4-fold increase in BTB permeability while sildenafil and vardenafil caused increases of 1.8-fold and 2.7-fold, respectively. Transport of the tracer in normal brain was not affected by the treatments (data not shown). Iberiotoxin, a selective K_{Ca} channel antagonist, abolished the vardenafil-induced BTB transport increase (FIG. 9B), suggesting that the effects by PDE5 inhibitors are mediated, at least in part, through the K_{Ca} channels.

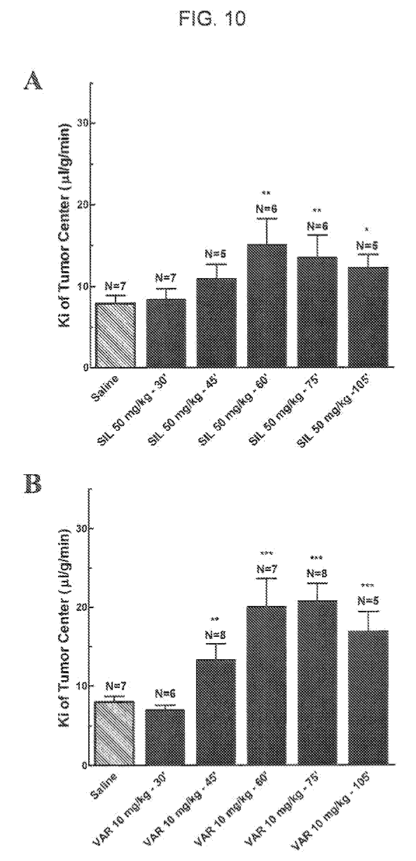

It was found that Ki values remained elevated between 60 and 105 minutes after oral administration of sildenafil (50 mg/kg) and 45 to 105 minutes after vardenafil (10 mg/kg) (FIGS. 10A and 10B). Transport across the BTB into tumor tissues reached the maximum at 60 and 75 minutes after administration of sildenafil and vardenafil, respectively. A much shorter duration (5-20 minutes) of Ki elevation has been reported for BK infusion (Nomura et al., Intracarotid infusion of bradykinin selectively increases blood-tumor permeability in 9L and C6 brain tumors. BRAIN RES. 1994; 659: 62-66; Inamura et al., Bradykinin selectively opens blood-tumor barrier in experimental brain tumors. J CEREB BLOOD FLOW METAB. 1994; 14: 862-870).

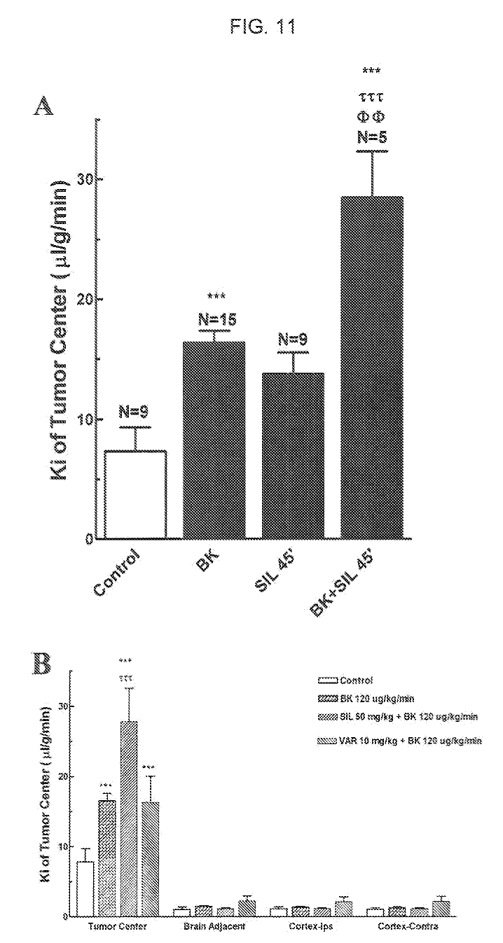

To determine any possible benefit of combination treatment, 9L tumor-bearing rats were given by gavage sildenafil or vardenafil with or without a 15-minute intravenous BK (120 μg/kg/min) infusion. The combination of BK and sildenafil treatment resulted in an increase in transport across the BTB at 45 minutes after the treatment as compared to either sildenafil or BK alone (p<0.001) (FIG. 11A). However, combining vardenafil with BK did not produce an increase in tumor transport (data not shown). The combination of sildenafil and BK did not increase transport in normal brain (FIG. 11B).

### Example 31

### Animal Physiologic Parameters

Mean-arterial blood pressures were decrease approximately 30% secondary to the femoral infusion of BK. The sildenafil (5-100 mg/kg) or vardenafil (1-20 mg/kg) caused a reduction in mean-arterial blood pressure of only 10%. Arterial blood pH, carbon dioxide, and partial pressure of oxygen were not changed significantly by the femoral infusion of BK or by the oral administration of sildenafil or vardenafil.

### Example 32

### cGMP Levels in the Plasma and in 9L Tumors of Rats after Oral Administration of PDE5 Inhibitors

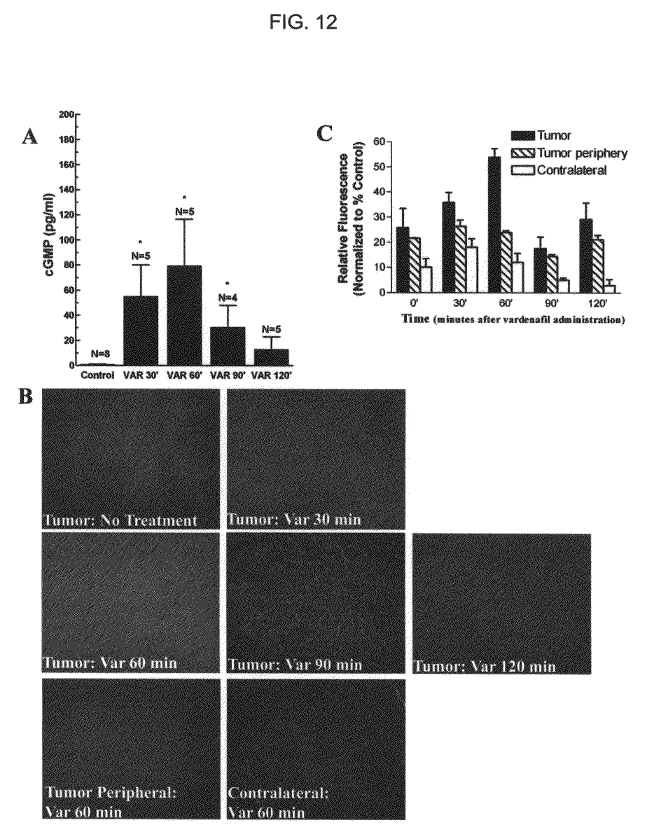

To test whether the effect of PDE5 inhibition Ki is related to cGMP signaling, the levels of cGMP in the plasma and tumor tissue from 9L tumor-bearing rats were measured. Plasma cGMP levels significantly increased at 30, 60, and 90 minutes (54.96±25.13 pg/ml, p<0.05; 79.20±37.36 pg/ml, p<0.05; 30.13±17.82 pg/ml, p<0.05, respectively) after oral administration of vardenafil as compared to no treatment controls (0.72±0.48 pg/ml), with the peak concentration at 60 minutes (FIG. 12A). Immunohistochemistry was performed to determine cGMP levels within the tumor (FIG. 12B). The semi-quantitative measurement of cGMP levels using Zeiss AxionVision software in untreated tumor-bearing rats showed that the normal brain contralateral to the tumor had very low levels of cGMP while the tumor tissue had increased cGMP-immunopositive staining (FIGS. 12B and 12C). Vardenafil treatment further increased immunostaining in the tumor tissues. The increase was apparent at 30 and 60 minutes after the drug treatment, and returned to the baseline at 90 minutes. Interestingly, although there was a trend for increase, no significant increase by vardenafil treatment in immunoflourescent signal for cGMP in normal brain contralateral to the tumor was observed. These results are consistent with those of transport studies, indicating that PDE5 inhibitors have selective effects on the transport across the BTB in tumors compared to the normal brain.

### Example 33

### Vesicular Density and Tight Junction Integrity in Brain Capillaries after Oral Administration of Vardenafil

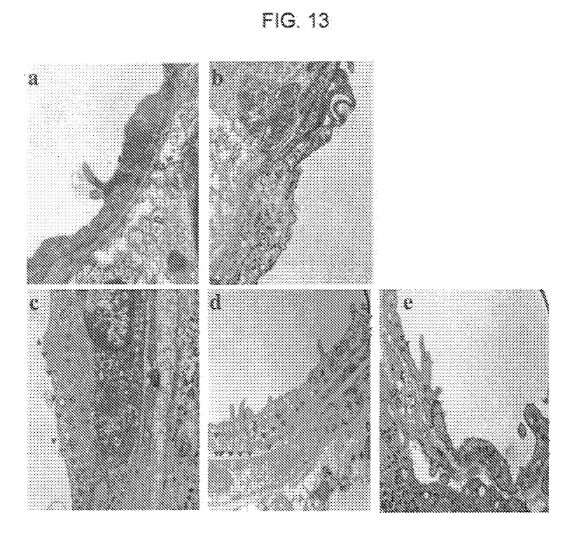

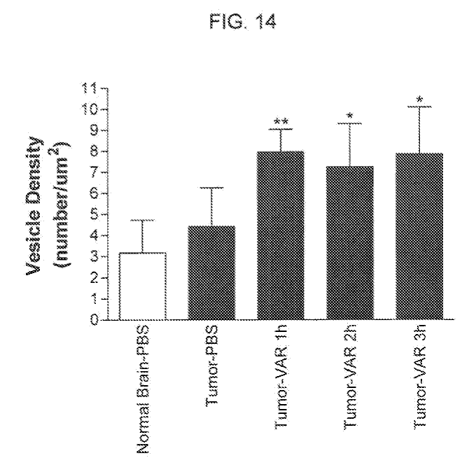

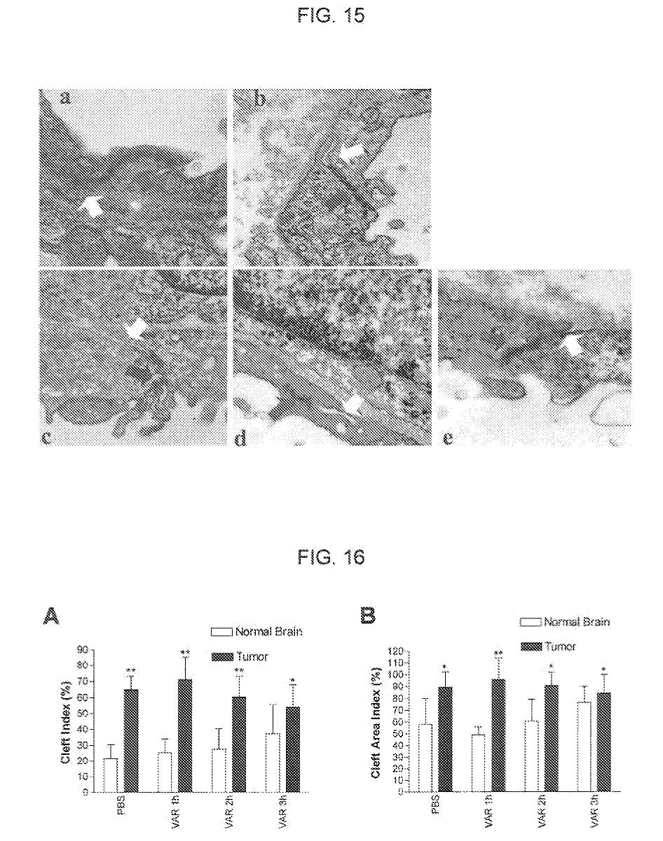

The inventor's previous studies (Ningaraj et al., Regulation of blood-brain tumor barrier permeability by calcium-activated potassium channels. J PHARMACOLOGY AND EXPERIMENTAL THERAPEUTICS. 2002; 301: 838-851; Hashizume et al., Increased endothelial vesicular transport correlated with increased blood-tumor barrier permeability induced by bradykinin and leukotrienes C4. J NEUROPATHOL EXP NEUROL 2002; 61: 725-735) indicate that increased vesicular transport is an important cellular mechanism for enhanced drug delivery via biochemical modulation (e.g., BK treatment) of BTB. Here, it was investigated vesicular density and tight junction integrity in BTB after oral treatment of vardenafil in rat tumor models. Vesicular formation was similar between normal and tumor capillary endothelium (FIG. 13). The vesicular formation was unchanged in the normal capillaries by vardenafil compared with PBS treated group (data not shown). However, vardenafil treatment dramatically increased vesicular formation in the tumor capillary endothelium (FIG. 13). This was further indicated by quantitative analysis of vesicle numbers in the capillary endothelium. The vesicular density and cumulative area of vesicles in vardenafil treated groups were significantly larger than those of PBS treated groups (FIG. 14). It was noticed that the effect by vardenafil on vesicular density lasted at least 3 hours. Reaction product for blood borne HRP was seen on the luminal surface membrane and within endothelial vesicles, endosomes, multivesicular bodies, and tumor cells nearby (data not shown). The tight junction integrity, which can be reflected by cleft index and cleft area (Ningaraj et al., Regulation of blood-brain tumor barrier permeability by calcium-activated potassium channels. J PHARMACOLOGY AND EXPERIMENTAL THERAPEUTICS. 2002; 301: 838-851; Ningaraj et al., Adenosine 5′-triphosphate-sensitive potassium channel-mediated blood-brain tumor barrier permeability increase in a rat brain tumor model. CANCER RES. 2003; 63: 8899-88911), in tumor capillary endothelium was worse compared to that in normal brain tissue (FIGS. 15 and 16). However, vardenafil treatment did not result in any changes in tight junction integrity.

### Example 34

### Survival Study

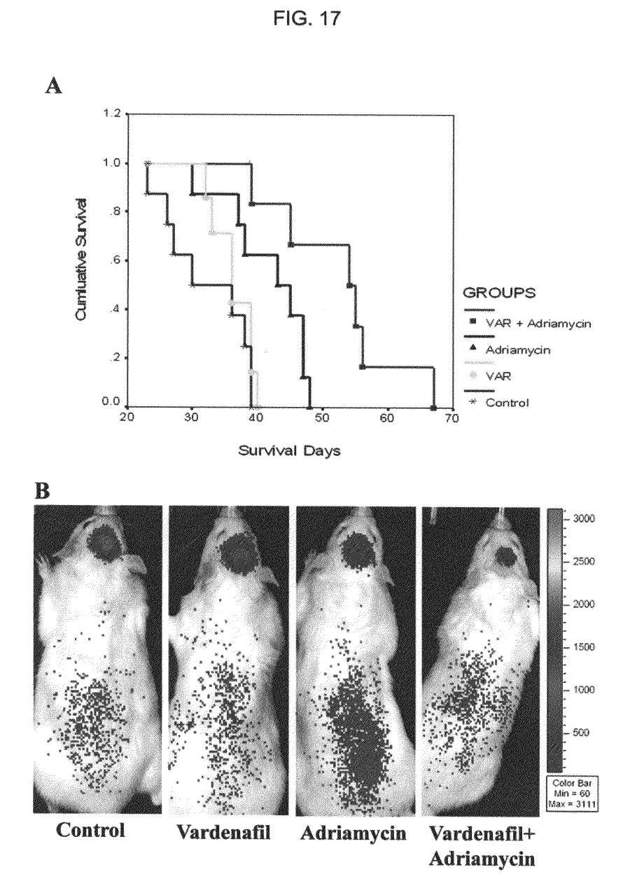

In order to determine if the permeability increase in brain tumors by the PDE5 inhibitors can be translated to improve efficacy of chemotherapy, the chemotherapeutic, Adriamycin® (doxorubicin), was administrated in 9L brain tumor-bearing rats, with and without vardenafil. Fischer rats were implanted intracranially with 1×10⁵ 9L cells, and treated for three days beginning at day 4 after tumor implantation with oral administration of vardenafil (10 mg/kg) followed by tail-vein injection of Adriamycin® (doxorubicin) (2 mg/kg). Log-rank analysis of the Kaplan-Meier survival curves showed a significant increase (p<0.05) in survival in the rats treated with vardenafil and Adriamycin® (doxorubicin) together as compared to the untreated, vardenafil alone- or Adriamycin® (doxorubicin) alone-treated rats (FIG. 17A). The mean survival time for the rats treated with vardenafil combined with Adriamycin® (doxorubicin) was significantly longer (53±4 days) compared to those of saline (32±2 days), vardenafil alone- (35±1 days) and Adriamycin® (doxorubicin) alone-treated rats (42±2 days). As expected, Adriamycin® (doxorubicin) alone also significantly increased the survival of 9L brain tumor bearing rats when compared to saline (p<0.05) (FIG. 17A).

In addition to monitoring survival, images of tumors in the rats were obtained and the tumor sizes were calculated by using the IVIS system at day 4 after tumor implantation and day 9 after the last treatment. At day 4 after implantation, the size of the tumor was similar among all of the 4 groups (data not shown). However, after 9 day treatment, the saline control, vardenafil-treated and Adriamycin® (doxorubicin)-treated groups had relatively larger intracerebral tumors [(2.21±1.27)×10⁶, (3.14±2.04)×10⁶, (1.03±0.28)×10⁶, respectively] compared to the group treated with the combination of vardenafil and Adriamycin® (doxorubicin) [(4.75±0.45)×10⁵] (FIG. 17B).

In a separate experiment 9 days after the last treatment, histology staining confirmed that the tumor sizes of the animals receiving the combination treatment were smaller than those of the other three groups. The tumor was more restricted for the combination treatment group (not shown). The animals in the combination treatment group showed no symptoms of dying while some animals in the other 3 groups showed neurological symptoms and started dying at the time of sacrifice.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method to selectively enhance the permeability of the blood-brain barrier of abnormal brain tissue (“abnormal BBB”) and/or the blood-tumor barrier (“BTB”) in a mammal in need thereof, comprising:
providing a 5-phosphodiesterase (“PDE5”) inhibitor; and
administering the PDE5 inhibitor in an amount sufficient to selectively enhance the permeability of the abnormal BBB and/or the BTB.

2. The method of claim 1, wherein the abnormal brain tissue is a brain tumor and the abnormal BBB and/or the BTB permeability is enhanced in the brain tumor.

3. The method of claim 1, wherein the BTB permeability is enhanced in a non-central nervous system (“CNS”) tumor.

4. The method of claim 3, wherein the non-CNS tumor is breast cancer, lung cancer or both.

5. The method of claim 1, wherein the PDE5 inhibitor is selected from the group consisting of sildenafil, vardenafil, salts thereof, analogs thereof and combinations thereof.

6. The method of claim 1, wherein the permeability of the abnormal BBB and/or the BTB is enhanced for at least 30 minutes.

7. The method of claim 1, wherein the permeability of the abnormal BBB and/or the BTB is enhanced for about 30 to about 360 minutes.

8. The method of claim 1, wherein the PDE5 inhibitor is orally administered.

9. The method of claim 1, wherein the PDE5 inhibitor is administered prior to administering a therapeutic agent or an imaging agent.

10. The method of claim 9, wherein the PDE5 inhibitor is administered about 30 to about 180 minutes prior to administering the therapeutic agent or the imaging agent.

11. The method of claim 10, wherein the PDE5 inhibitor is administered about 30 to about 45 minutes prior to administering the therapeutic agent or the imaging agent.

12. The method of claim 9, wherein the therapeutic agent is an anti-cancer drug.

13. The method of claim 12, wherein the anti-cancer drug is doxorubicin, carboplatin or both.

14. The method of claim 1, further comprising:
administering an additional agent that enhances the permeability of the abnormal BBB and/or the BTB selected from the group consisting of a bradykinin, a nitrogen oxide donor drug, a potassium channel agonist, analogs thereof, salts thereof and combinations thereof.

15. A method for treating abnormal brain tissue and/or a tumor in a mammal in need thereof, comprising:
providing a 5-phosphodiesterase (“PDE5”) inhibitor;
administering the PDE5 inhibitor in an amount sufficient to selectively enhance the permeability of the blood-brain barrier of abnormal brain tissue (“abnormal BBB”) and/or the blood-tumor barrier (“BTB”) in the mammal; and
administering a therapeutic agent to the mammal.

16. The method of claim 15, wherein the abnormal brain tissue is selected from the group consisting of a brain tumor, a tissue affected by a disease, a tissue affected by a physical injury, and combinations thereof.

17. The method of claim 16, wherein the brain tumor is selected from the group consisting of glioma, glioblastoma, glioblastoma multiforme, gliosarcoma, oligodendroglioma, primitive neuroectodermal tumor, astrocytoma, ependymoma, oligodendroglioma, medulloblastoma, meningioma, pituitary adenomas, neuroblastoma, craniopharyngioma and combinations thereof.

18. The method of claim 15, wherein the tumor is a non-central nervous system (“CNS”) tumor.

19. The method of claim 18, wherein the non-CNS tumor is breast cancer, lung cancer, or both.

20. The method of claim 15, wherein the PDE5 inhibitor is selected from the group consisting of sildenafil, vardenafil, salts thereof, analogs thereof and combinations thereof.

21. The method of claim 15, wherein the PDE5 inhibitor is administered prior to administering the therapeutic agent.

22. The method of claim 15, wherein the PDE5 inhibitor is administered about 30 to about 180 minutes prior to administering the therapeutic agent.

23. The method of claim 15, wherein the PDE5 inhibitor is administered about 30 to about 45 minutes prior to administering the therapeutic agent.

24. The method of claim 15, wherein the PDE5 inhibitor is orally administered.

25. The method of claim 15, wherein the therapeutic agent is doxorubicin, carboplatin or both.

26. The method of claim 15, further comprising:
administering an additional agent that enhances the permeability of the abnormal BBB and/or the BTB selected from the group consisting of a bradykinin, a nitrogen oxide donor drug, a potassium channel agonist, salts thereof, analogs thereof and combinations thereof.

27. A kit for the treatment of a tumor in a mammal in need thereof, comprising:
a quantity of a PDE5 inhibitor to selectively enhance the permeability of the blood-brain barrier of abnormal brain tissue (“abnormal BBB”) and/or the blood-tumor barrier (“BTB”);
a quantity of an anti-cancer drug; and
instructions for using the PDE5 inhibitor to selectively enhance the permeability of the abnormal BBB and/or the BTB to the anti-cancer drug to treat the tumor.
